(19) United States
(12) Patent Application Publication
(10) Pub. No.: US 2009/0208446 A1
(43) Pub. Date: Aug. 20, 2009

(54) DUB3 as a Cancer Therapy Target

(75) Inventors: James Johnston, Belfast (GB); James Burrows, Belfast (GB); Alyson Kelvin, London (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: THE QUEEN'S UNIVERSITY OF BELFAST, Belfast (GB)

(21) Appl. No.: 12/227,258

(22) PCT Filed: May 14, 2007
(86) PCT No.: PCT/GB2007/050262

§ 371 Date: Apr. 2, 2009

(30) Foreign Application Priority Data
May 12, 2006 (GB) 0609414.8
Mar. 1, 2007 (GB) 0703963.9

(51) Int. Cl. A61K 31/7088; C12N 9/99; C12Q 1/37; A61K 38/19; C12Q 1/68

(52) U.S. Cl. 424/85.1; 435/184; 435/23; 435/6

(57) ABSTRACT

Described is a method of modulating the activation of a Rho, Arf, Rab, Ran or Rap family protein, said method comprising administering to said sample a USP-17 modulator, for example an inhibitor of expression or activity of DUB-3. The invention enables the use of USP-17 modulators in the treatment of metastatic cancer and other conditions.

### FIELD OF THE INVENTION

This application relates to methods of treatment of inflammatory diseases, infectious diseases and neoplastic diseases and compositions for use in such treatments. In particular, it relates to methods of inhibiting or treating disease associated with or consequent to cell invasion, such as the development of metastases in an animal having a primary tumour, methods of reducing acute and chronic inflammation and methods of reducing the spread of infection throughout the body.

### BACKGROUND TO THE INVENTION

Metastatic spread of disease is a poor prognostic factor in the treatment of cancer. Although many existing chemotherapeutic strategies have some success in the treatment in primary tumours, such strategies are often less successful in the prevention of formation of metastases and the treatment of such metastases when they have arisen. There therefore remains a pressing need to develop new strategies which protect against the development of metastases from primary tumours and which eradicate metastases when and where they arise.

Deubiquitination, the removal of monomers or chains of ubiquitin from linked proteins, is carried out by cysteine proteases that can be divided into 2 main subfamilies: the ubiquitinatin-processing proteases (UBPs) and the ubiquitin carboxyl-terminal hydrolases (UCHs) (reviewed by Wing 2003) The DUB, deubiquitinating enzyme, family of deubiquitinating enzymes belongs to the ubiquitin specific processing proteases (USP) subfamily USP-17 and the first member was identified specifically expressed in hematapoietic cells (Zhu et al., 1996a). Since then, four more DUB family members have been identified including a human member Dub-3 by Burrows and colleagues in 2004 (Zhu et al., 1997; Baek et al., 2004; Baek et al., 2001; Burrows et al., 2004; Saiton et al., 2000). Dub-3 is expressed in various malignant cell lines suggesting a potential role for Dub-3 in cancer pathogenesis (Burrows et al., 2004). Previously, other deubiquitinating enzymes have been shown to play a role in oncogenic pathways. The deubiquitinating enzymes Unp (ubiquitous nuclear protein), VDU2 (pVHL-interacting deubiquitinating enzyme-2) and HAUSP (herpes virus-associated ubiquitin-specific protease) have been shown to interact with the tumour suppressors Rb (retinoblastoma protein), pVHL (von Hippel-Lindau) and p53, respectively (DeSalle L M et al., 2001; Li Z. et al., 2002; Li M et al., 2002).

Recently, it has been found that the activity of Ras superfamily members can be regulated by posttranslational processing and not just by GTP-GDP cycling. Members of the Ras superfamily have a C-terminal CAAX motif (A is an aliphatic amino acid and X is any amino acid) and the posttranslational modifications to this C-terminal CAAX motif are responsible for an alternative mode of GTPase activation. The posttranslational modifications are achieved by the actions of a series of enzymes: a cytosolic prenyltransferase, an endoplasmic reticulum (ER) protease RCE1 (Ras converting enzyme), and the ER enzyme ICMT (isoprenylcysteine-directed carboxylmethyltransferase) (Clark 1992; Casey and Seabra 1996; Boyartchuk et al., 1997; Dia et al., 1998). The product of these posttranslational modifications is a hydrophobic domain at the C-terminus of a hydrophilic GTPase molecule that eventually leads to full GTPase activation.

The Ras superfamily of 20-30 kDa monomeric guanine nucleotide binding proteins has over 150 known human members and can be divided into 5 families: Ras, Rho, Arf, Rab, and Ran (Wennerberg et al., 2005), the members of each of which has different effects on cells and is subject to different regulatory influences. For example, Ras regulates proliferation via growth factors; the Rho family does not. Further, although Ras is regulated by farnesylation, not all members of the Ras family are regulated by farnesylation. For instance Rap-1 is regulated by geranylgeranylation. Moreover, the Rho family of proteins is regulated by geranylgeranylation. Also the Rho family regulates the actin cytoskeleton and Rap-1 regulates adhesion; Ras does not.

The Rho family is most associated with cellular movement and tumour metastasis. Of the Rho family, RhoA, Rac-1 and Cdc42 are the GTPases most associated with cancer invasion. Cancer cell invasion and metastasis are regulated by three cellular processes: cell adhesion, cell migration and the production of extra cellular matrix degrading enzymes and these three processes are all driven by Ras superfamily GTPases (Katagiri et al., 2000; Eden et al., 2002; Zhuge et al., 2001, respectively).

As described in WO2005/049818, the present inventors have identified Dub-3 as a regulator of Ras processing and activation, possibly via regulation of Rce1. In particular, this document discloses that agents which increase DUB-3 may be advantageous in decreasing activation of Ras and that such agents may be used in the treatment of cancer.

As described above, the different families of the Ras superfamily differ greatly from each other in many aspects, including their regulation and their effects. Accordingly, the identification of a regulatory pathway for one family of the Ras superfamily does not indicate that such a regulatory pathway applies to other families of the superfamily.

### SUMMARY OF THE INVENTION

As described herein, despite the many differeneces between the regulation of families of the Ras superfamily, the present inventors have unexpectedly found that the USP-17 enzyme Dub-3 plays a role in the regulation of Rho proteins. Further surprising was the demonstration that both up and down regulation of DUB3 has the effect of increasing activation of Rho proteins.

Moreover, the inventors have further shown that Dub-3 plays a role in chemokine stimulated cell adhesion, migration and chemoinvasion of malignant cells through the modulation of Rho proteins and that, as a result, USP-17 enzymes such as DUB-3 may be used as a therapeutic target for the treatment of cancer metastasis and invasion.

Accordingly, in a first aspect of the present invention, there is provided a method of modulating the activation of a Rho protein in a biological sample, said method comprising administering to said sample a USP-17 modulator.

The method of the invention may be used to modulate activation of any Rho protein, for example RhoA, RhoB, RhoC, Rac1, Rac2, Rac3, RhoG, Cdc42, TC10, TCL, Wrch-1, Rnd1, Rnd2, RhoE/Rnd3, RhoD, Rif, and/or RhoH/TIF.

In one embodiment, the Rho protein is Rac-1.

Moreover, the demonstration that, contrary to expectations, Dub-3 affects the regulation of Rho proteins suggests that USP-17 enzymes, for example Dub-3, will play a regulatory role over other families of the Ras superfamily other than the Ras family.

Thus, in a second aspect of the present invention, there is provided a method of modulating the activation of a Ras superfamily protein in a biological sample, said method comprising administering to said sample an agent which modulates a USP-17 enzyme, wherein said Ras superfamily protein is a Rho, Arf, Rab, or Ran protein.

In another aspect of the invention, there is provided a method of modulating the activation of a Ras superfamily protein in a biological sample, said method comprising administering to said sample an agent which modulates a USP-17 enzyme, wherein said Ras superfamily protein is a Ral, Rap or Rheb protein. In one embodiment, the Ras superfamily protein is selected from the group comprising RalA, RalB, Rap1a, Rap1b, Rap2a, Rap2b, and Rheb.

The inventors have investigated the control of DUB-3 in order to elucidate its role in vivo. As described in the Examples, the inventors have found that DUB-3 is regulated at the message level by the chemokine, CXCL12/SDF. Thus in one embodiment of the first or second aspect of the invention, the activation of the Ras superfamily protein, for example Rho family protein, is chemokine stimulated activation.

Further, in a third aspect of the present invention, there is provided a method of stimulating expression of USP-17 enzymes, for example DUB-3, in a cell, said method comprising administering to said cell a chemokine.

Any suitable chemokine may be used. Suitable chemokines include, but are not limited to, IL-8, RANTES, MIP-1α, MIP-1b, MCP-1, CCL19, CCL21 or a G-Protein Coupled Receptor Chemoattractant Ligand.

In one embodiment the chemokine is CXCL12/SDF Chemokine stimulation is known to induce cell adhesion, cell migration and cell invasion (chemoinvasion). Since chemokine stimulation was found to regulate Dub-3 message expression, the inventors investigated if deregulating Dub-3 could affect chemokine driven functions.

As described in the Examples, it was surprisingly shown that the modulation of Dub3 inhibits CXCL12/SDF stimulated chemotaxis, and CXCL12/SDF stimulated migration and invasion through an artificial basement membrane.

Accordingly, the inventors have established that USP 17 enzymes, such as Dub3, are important modulators of chemokine driven cellular events and are potential therapeutic targets for diseases and conditions mediated by cell invasion, such as inflammatory and infectious diseases associated with cell invasion, and tumour metastasis.

Thus, according to a fourth aspect of the present invention, there is provided a method of modulating chemokine induced cell invasion, said method comprising administering a USP-17 modulator.

In a fifth aspect, there is provided a method of modulating chemokine induced cell adhesion, said method comprising administering a USP-17 modulator.

In a sixth aspect of the invention, there is provided a method of modulating chemokine induced cell migration, said method comprising administering a USP-17 modulator.

In one embodiment of the fourth, fifth or sixth aspects of the invention, the chemokine is CXCL12.

The inventors investigated the effect of Dub-3 on a number of Rho proteins, such as Rac, RhoA and Cdc42 and on Rap1 and, as described in the Examples, demonstrated that DUB-3 modulation deregulates Rac and Rap 1 activation and inhibits SDF-1/CXCL12 stimulated plasma membrane translocation of both proteins and, further, inhibits cell proliferation.

Accordingly, in a seventh aspect of the invention, there is provided a method of inhibiting chemokine induced translocation of a Rho protein and/or a Rap protein in a chemotactic cell, said method comprising administering a USP-17 modulator to said cell.

In one embodiment, the Rho protein is Rac.

In one embodiment, the chemokine is SDF-1/CXCL12.

By enabling the modulation of cell migration and invasion, the invention finds use in the treatment and prevention of metastasis.

Thus, in an eighth aspect of the invention there is provided a method of reducing the number of tumour metastases in an animal with a primary tumour, said method comprising administering a USP-17 modulator to said animal.

In the eighth aspect of the invention, the incidence of metastasis may be reduced i.e. the development of metastases may be inhibited, and/or one or more metastases may be eradicated such that the number of pre-existing metastases may be reduced.

Thus the invention may be used to treat stage 3 or Stage 4 tumours.

As well as being relevant to the treatment of tumour metastasis, the discovery that USP-17 deregulation may reduce cell invasion and migratory behaviour is also relevant to cells involved in inflammatory diseases characterised by cell migration.

Accordingly, in a ninth aspect of the present invention, there is provided a method of treating an inflammatory disease or infectious disease associated with cell invasion in an animal in need thereof, said method comprising Administering a USP-17 modulator to said animal.

Inflammatory diseases or conditions for which the invention may be used include rheumatoid arthritis, allograft rejection, diabetes, multiple sclerosis (MS)/experimental autoimmune encephalomyelitis (EAE), systemic lupus erythematosus (SLE), dermatitis, and asthma.

Other examples of inflammatory diseases or conditions for which the invention may be used include viral infections and bacterial infections including hepatitis, osteoarthritis, tuberculosis, respiratory infection, psoriasis, HIV, influenza, SARS, conjunctitis, shingles, meningitis, contact dermatitis, gingivitis, cellulites, pneumonia, inflammatory bowel disease, Crohns disease, ulcerative Colitis, skin ulceration, fungal infections, thrush, encephalitis, and urinary tract infections.

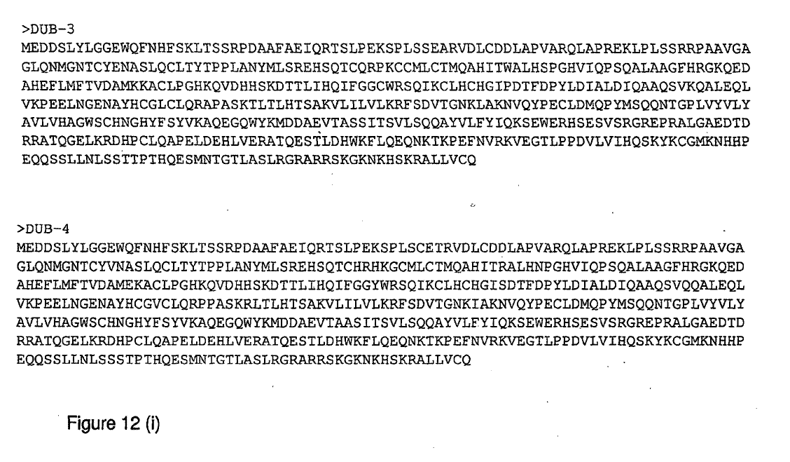

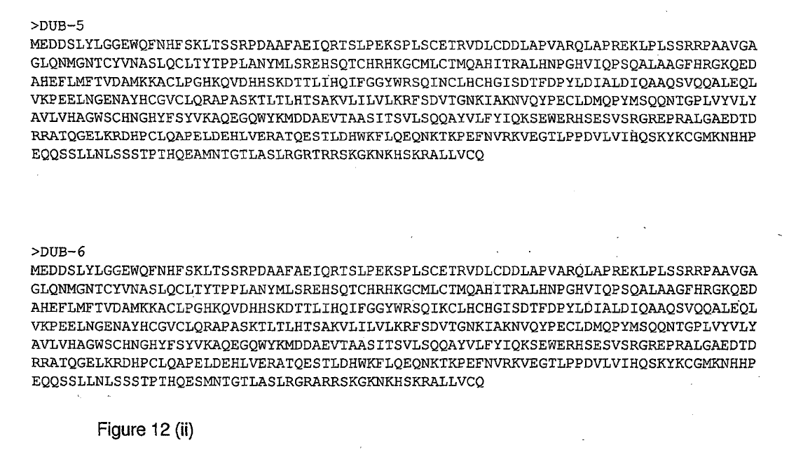

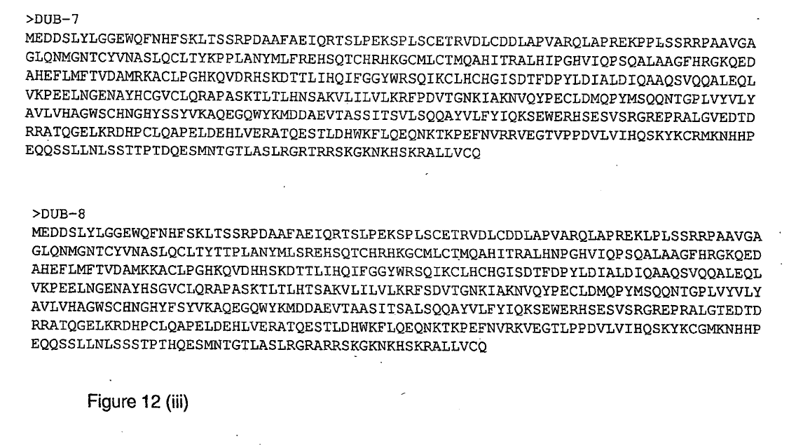

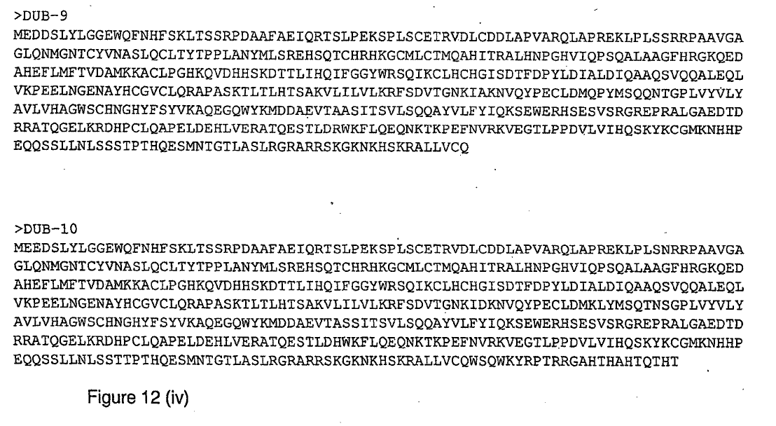

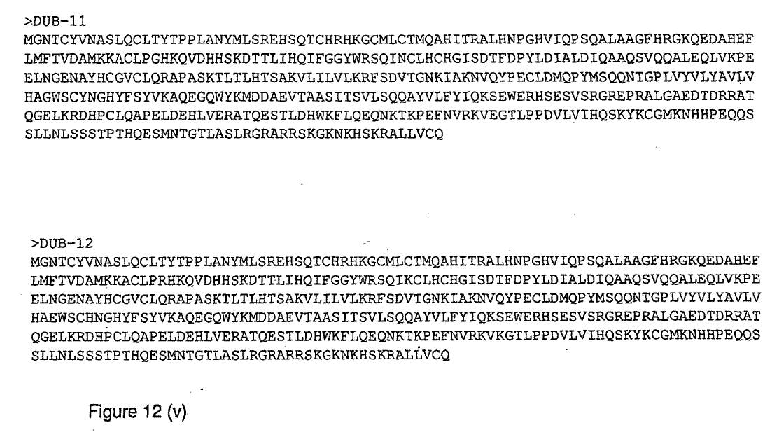

In one embodiment of the invention, the USP-17 enzyme is DUB-3. Other USP-17 enzymes, the modulation of which, may find use in the present invention, include USP-17 homologues, DUB-3, DUB-4, DUB-5, DUB-6, DUB-7, DUB-8, DUB-9, DUB-10, DUB-11, DUB-12, DUB-13, and DUB-14, and variants or fragments thereof. The amino acid sequences of each of DUB-3 to DUB-12 are shoen in FIG. 12. Further details relating to Dub-3 to Dub-12, as well as variants and fragments thereof, are described in WO 2005/049818, the contents of which are herein incorporated by reference.

Any suitable USP-17 modulators may be used in the present invention. The USP-17 modulators may modulate expression and/or activity of the USP-17 enzyme. The USP-17 modulators may result in an increase or decrease in one or more USP-17 enzymes compared to normal conditions. In one embodiment, the USP-17 modulator reduces the concentration of a particular USP-17 enzyme, for example DUB-3 protein in the cells to which it is administered. In another embodiment, the USP-17 modulator increases the concentration of one or more USP-17 proteins in the cells to which it is administered.

Modulators of USP-17 which may be used in the invention include, for example, small molecule agents, peptides or antibodies, nucleic acid molecules encoding said peptides or antibodies, aptamers, antisense molecules or siRNA molecules, for example a pSuper targeting construct against DUB3.

As described above and in the Examples, the inventors have shown that, in particular contrast to the results described in WO2005/049818, in which agents which increase DUB-3 were disclosed as advantageous in decreasing activation of Ras and that such agents may be used in the treatment of cancer, the down regulation of DUB-3 results in enhanced activation of Rho and Rap proteins and moreover inhibits cell processes such as chemotaxis, which are associated with pathologies such as metastatic cancer. Accordingly, in one embodiment, the USP-17 modulator inhibits activation or expression of one or more USP 17 enzymes, for example of DUB-3.

Thus the USP-17 modulator may be a USP-17 inhibitor such as a small molecule inhibitor such as a USP17 siRNA, for example DUB3 siRNA or an antibody molecule, such as an antibody or antibody fragment, for example, an antibody as disclosed in Burrows et al., 2004 (Burrows J F et al., JBC 2004 279 (14): 13993-4000).

An example of a hairpin siRNA sequence which may be used to inhibit DUB-3 is GCAGGAAGATGCCCATGAA TTCATGGGCATCTTCCTGC. Any suitable vector may be used. For example, in one embodiment, a pSUper targeting construct may be used.

The discovery of the link between USP-17 enzymes and Rho, Arf, Rab, and Ran protein activation and the effect of USP-17 enzymes on chemokine induced cell invasive and migratory behaviour further enables the identification of further modulators of activation of Rho, Arf, Rab, or Ran proteins and thus of modulators of cell invasion and migratory behaviour.

Accordingly, in a tenth aspect of the invention, there is provided an assay method for identifying a modulator of activation of a Rho, Arf, Rab, or Ran protein said method comprising the steps of:

a) bringing a candidate agent into contact with test cells capable of expressing a USP-17 protein,
b) determining the expression of the USP-17 protein in the presence of the candidate agent in said test cells,
c) comparing the expression of the USP-17 protein in the presence of the candidate agent with control cells not exposed to said candidate agent,
wherein a difference in expression of the USP-17 protein between the control cells and the test cells is indicative that the candidate agent may modulate activation of a Rho, Arf, Rab, or Ran protein.

Also provided in an eleventh aspect of the present invention is an assay method for identifying a modulator of tumour metastasis, said method comprising the steps of:

a) bringing a candidate modulator into contact with test cells capable of expressing a USP-17 protein,
b) determining the expression of said USP-17 protein in the presence of the candidate modulator in said test cells,
c) comparing the expression of the USP-17 protein in the presence of the candidate modulator with control cells not exposed to said candidate modulator; wherein a difference in expression of the USP-17 protein between the control cells and the test cells is indicative that the candidate modulator may be a modulator of tumour metastasis.

The assay method of the tenth or eleventh aspects of the invention may include confirmatory steps:

(d) providing an assay for determining activation of a Rho, Arf, Rab, or Ran family protein, for example Rac;
(e) determining the activation of the family protein in the presence and absence of the candidate modulator, wherein a decrease in the expression of the family protein in the presence of the candidate modulator compared to the expression of the family protein in the absence of the candidate modulator confirms the candidate agent as being an inhibitor of expression of the Rho, Arf, Rab, or Ran protein.

Furthermore, the assay of the eleventh aspect, as well as optionally including step (d) and (e) as described above, may, alternatively or additionally, comprise one or more further confirmatory assay steps.

In one embodiment, the further confirmatory assay step comprises:

(i) providing a cell migration assay;
(ii) determining migration of cells in the presence of the candidate modulator;
(iii) determining migration of cells in the absence of the candidate modulator; wherein a decrease in cell migration in the presence of said candidate modulator confirms the modulator as an inhibitor of tumour metastasis.

Another further confirmatory assay step, which may be used comprises:

(i) providing a cell adhesion assay;
(ii) determining cell adhesion of cells in the presence of the candidate modulator;
(iii) determining cell adhesion of cells in the absence of the candidate modulator; wherein a decrease in cell adhesion in the presence of said candidate modulator confirms the modulator as an inhibitor of tumour metastasis.

Another further confirmatory assay step which may be used comprises:

(i) providing a cell chemoinvasion assay;
(ii) determining chemoinvasion of cells in the presence of the candidate modulator;
(iii) determining chemoinvasion of cells in the absence of the candidate modulator; wherein a decrease in chemoinvasion in the presence of said candidate modulator confirms the modulator as an inhibitor of tumour metastasis.

A twelfth aspect of the invention provides an assay method for identifying a modulator of tumour GTPase translocation in a chemotactic cell, said method comprising the steps of:

a) bringing a candidate modulator into contact with test cells capable of expressing a USP-17 protein,
b) determining the expression of said USP-17 protein in the presence of the candidate modulator in said test cells,
c) comparing the expression of the USP-17 protein in the presence of the candidate modulator with control cells not exposed to said candidate modulator,
wherein a difference in expression of the USP-17 protein between the control cells and the test cells is indicative that the candidate modulator may be a modulator of GTPase translocation.

In one embodiment of the tenth to twelfth aspects of the invention, the USP-17 protein is DUB-3.

In one embodiment of this aspect of the invention, the GTPase is a Rho protein, for example Rac. In another embodiment, the GTPase is Rap1.

In one embodiment, the translocation is chemokine, cytokine or growth factor induced translocation. In one embodiment, the chemokine is SDF-1/CXCL12.

Also provided as a thirteenth aspect of the present invention is the use of a USP-17 modulator in the preparation of a medicament for the treatment of an inflammatory disease associated with cell migration.

A fourteenth aspect of the invention provides the use of a USP-17 modulator in the preparation of a medicament for the treatment of tumour metastasis and invasion.

In one embodiment of the thirteenth or fourteenth aspects of the invention, the USP-17 modulator decreases the expression or activity of the USP-17 protein.

The invention may be used in the treatment or prevention of metastasis of any type of tumour. For example, primary tumours, the metastases of which the present invention may be used to treat, include but are not limited to lung, colorectal, prostate, ovarian, lymphoma, breast, prostate, pancreatic, brain, bone, bladder, spleen cancers and head and neck tumours.

A fifteenth aspect of the invention provides the use of a DUB-3 modulator in the preparation of a medicament for the treatment of inflammatory disease.

As described in the Examples, the inventors have also found that DUB-3 modulates cytokine induced activation of Rap-1.

Accordingly, in a further aspect of the invention, there is provided a method of modulating the activation of a Rap protein in a biological sample, said method comprising administering to said sample a USP-17 modulator, for example DUB-3. In one embodiment of this aspect, the activation of Rap is chemokine, cytokine or growth factor induced activation.

In one embodiment of this aspect of the invention, the modulator increases the expression or activity of the USP-17 protein, for example DUB-3.

Preferred features of each aspect of the invention are as for each of the other aspects mutatis mutandis.

### DETAILED DESCRIPTION

### USP-17 Modulators

Any suitable USP-17 modulator may be used in the present invention. The USP-17 modulator may increase or decrease activity or expression of USP-17.

As described above, the modulators may be nucleic acid molecules or antibody molecules. In another embodiment, the USP-17 modulator may be a peptide or non-peptide small molecule modulator of DUB-3. In a further embodiment the USP-17 modulator may be an aptamer. For example, the modulator may be siRNA, for example using the pSUPER RNAi system (Oligoengine, Seattle, USA). The pSUPER RNAi system is a mammalian expression vector that directs intracellular synthesis of siRNA-like transcripts. The vector uses the polymerase-III H1-RNA gene promoter.

In one embodiment, the USP-17 modulator is a DUB-3 modulator. In a particular embodiment, the DUB-3 modulator for use in the invention is a DUB-3 inhibitor.

### Nucleic Acid

Nucleic acid for use in the present invention may comprise DNA or RNA. It may be produced recombinantly, synthetically, or by any means available to those in the art, including cloning using standard techniques.

The nucleic acid may be inserted into any appropriate vector, for example a virus (e.g. vaccinia virus, adenovirus, etc.), baculovirus; yeast vector, phage, chromosome, artificial chromosome, plasmid, or cosmid DNA. Vectors may be used to introduce the nucleic acids into a host cell, which may be prokaryotic or eukaryotic.

For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 5th ed., Ausubel et al. eds., John Wiley & Sons, 2005 and, Molecular Cloning: a Laboratory Manual: 3^{rd} edition Sambrook et al., Cold Spring Harbor Laboratory Press, 2001.

### Antisense/siRNA

USP-17 modulators for use in the present invention may comprise nucleic acid-molecules capable of modulating gene expression, for example capable of down regulating expression of a sequence encoding a DUB-3 protein. Such nucleic acid molecules may include, but are not limited to antisense molecules, short interfering nucleic acid (siNA), for example short interfering RNA (siRNA), double-stranded RNA (dsRNA), micro RNA, short hairpin RNA (shRNA), nucleic acid sensor molecules, allozymes, enzymatic nucleic acid molecules, and triplex oligonucleotides and any other nucleic acid molecule which can be used in mediating RNA interference “RNAi” or gene silencing in a sequence-specific manner (see for example Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914; Allshire, 2002, Science, 297, 1818-1819; Volpe et al., 2002, Science, 297, 1833-1837; Jenuwein, 2002, Science, 297, 2215-2218; Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831).

An “antisense nucleic acid”, is a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). The antisense molecule may be complementary to a target sequence along a single contiguous sequence of the antisense molecule or may be in certain embodiments, bind to a substrate such that the substrate, the antisense molecule or both can bind such that the antisense molecule forms a loop such that the antisense molecule can be complementary to two or more non-contiguous substrate sequences or two or more non-contiguous sequence portions of an antisense molecule can be complementary to a target sequence, or both. Details of antisense methodology are known in the art, for example see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

A “triplex nucleic acid” or “triplex oligonucleotide” is a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504).

### Aptamers

Aptamers are nucleic acid (DNA and RNA) macromolecules that bind tightly to a specific molecular target. They can be produced rapidly through repeated rounds of in vitro selection for example by SELEX (systematic evolution of ligands by exponential enrichment) to bind to various molecular targets such as small molecules, proteins, nucleic acids etc (see Ellington and Szostak, Nature 346(6287):818-822 (1990), Tuerk and Gold, Science 249(4968):505-510 (1990) U.S. Pat. No. 6,867,289; U.S. Pat. No. 5,567,588, U.S. Pat. No. 6,699,843).

In addition to exhibiting remarkable specificity, aptamers generally bind their targets with very high affinity; the majority of anti-protein aptamers have equilibrium dissociation constants (Kds) in the picomolar (pM) to low nanomolar (nM) range.

Aptamers are readily produced by chemical synthesis, possess desirable storage properties, and elicit little or no immunogenicity in therapeutic applications.

Non-modified aptamers are cleared rapidly from the bloodstream, with a half-life of minutes to hours, mainly due to nuclease degradation and renal clearance a result of the aptamer's inherently low molecular weight. However, as is known in the art, modifications, such as 2′-fluorine-substituted pyrimidines, polyethylene glycol (PEG) linkage, etc. (can be used to adjust the half-life of the molecules to days or weeks as required.

Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein which has good solubility and compacity properties. Aptamers may comprise any deoxyribonucleotide or ribonucleotide or modifications of these bases, such as deoxythiophosphosphate (or phosphorothioate), which have sulfur in place of oxygen as one of the non-bridging ligands bound to the phosphorus. Monothiophosphates αS have one sulfur atom and are thus chiral around the phosphorus center.

Dithiophosphates are substituted at both oxygens and are thus achiral. Phosphorothioate nucleotides are commercially available or can be synthesized by several different methods known in the art.)

### Antibody Molecules

In the context of the present invention, an “antibody molecule” should be understood to refer to an immunoglobulin or part thereof or any polypeptide comprising a binding domain which is, or is homologous to, an antibody binding domain. Antibodies include but are not limited to polyclonal, monoclonal, monospecific, polyspecific antibodies and fragments thereof and chimeric antibodies comprising an immunoglobulin binding domain fused to another polypeptide.

Intact (whole) antibodies comprise an immunoglobulin molecule consisting of heavy chains and light chains, each of which carries a variable region designated VH and VL, respectively. The variable region consists of three complementarity determining regions (CDRs, also known as hypervariable regions) and four framework regions (FR) or scaffolds. The CDR forms a complementary steric structure with the antigen molecule and determines the specificity of the antibody.

Fragments of antibodies may retain the binding ability of the intact antibody and may be used in place of the intact antibody. Accordingly, for the purposes of the present invention, unless the context demands otherwise, the term “antibodies” should be understood to encompass antibody fragments. Examples of antibody fragments include Fab, Fab′, F (ab′)₂, Fd, dAb, and Fv fragments, scFvs, bispecific scFvs, diabodies, linear antibodies (see U.S. Pat. No. 5,641,870, Example 2 Zapata et al., Protein Eng 8 (10): 1057-1062 [1995]); single-chain antibody molecules; and multispecific antibodies formed from antibody fragments.

The Fab fragment consists of an entire L chain (VL and CL), together with VH and CH1. Fab′ fragments differ from Fab fragments by having additional few residues at the carboxy terminus of the CH1 domain including one or more cysteines from the antibody hinge region. The F (ab′) 2 fragment comprises two disulfide linked Fab fragments.

Fd fragments consist of the VH and CH1 domains.

Fv fragments consist of the VL and VH domains of a single antibody.

Single-chain Fv fragments are antibody fragments that comprise the VH and VL domains connected by a linker which enables the scFv to form an antigen binding site. (see Pluckthun in The Pharmacology of Monoclonal Antibodies, vol. 113, Rosenburg and Moore eds., Springer-Verlag, New York, pp. 269-315 (1994). Diabodies are small antibody fragments prepared by constructing scFv fragments (see preceding paragraph) with short linkers (about 5-10 residues) between the VH and VL domains such that inter-chain but not intra-chain pairing of the V domains is achieved, resulting in a multivalent fragment, i.e. a fragment having two antigen-binding sites (see, for example, EP 404 097; WO 93/11161; and Hollinger et al., Proc. Natl. Acad. Sci. USA, 90′: 6444-6448 (1993))

Further encompassed by fragments are individual CDRs.

In one embodiment of the present invention, the USP-17 modulator is an antibody molecule which inhibits the proteolytic activity or deubiquitinating activity of DUB-3. Examples of such suitable antibodies are disclosed in Burrows J F et al., JBC 2004 279 (14): 13993-4000.

As described above, the antibody molecules for use in the present invention extends, for example, to any other antibody which inhibits deubiquitinating activity. For example variants of known anti Dub-3 antibodies, which maintain the ability to inhibit deubiquitinating activity may be used. Thus, the CDR amino acid sequences of known USP-17, for example DUB-3 antibody molecules in which one or more amino acid residues are modified, may also be used as the CDR sequence. Such variants may be provided using techniques known in the art. The CDRs may be carried in a framework structure comprising an antibody heavy or light chain sequence or part thereof. Preferably such CDRs are positioned in a location corresponding to the position of the CDR(s) of naturally occurring VH and VL domains. The positions of such CDRs may be determined as described in Kabat et al, Sequences of Proteins of Immunological Interest, US Dept of Health and Human Services, Public Health Service, Nat'l Inst. of Health, NIH Publication No. 91-3242, 1991 and online at www.kabatdatabase.com http://immuno.bme.nwu.edu.

Furthermore, modifications may alternatively or additionally be made to the Framework Regions of the variable regions. Such changes in the framework regions may improve stability and reduce immunogenicity of the antibody.

Antibodies for use in the invention herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human.primate (e.g. Old World Monkey, Ape etc), and human constant region sequences.

Antibody molecules for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352: 624-628 and Marks et al. (1992) Bio/Technology, 10 779-783). Other antibody production techniques are described in Antibodies: A Laboratory Manual, eds. Harlow et al., Cold Spring Harbor Laboratory, 1988.

Traditional hybridoma techniques typically involve the immunisation of a mouse or other animal with an antigen in order to elicit production of lymphocytes capable of binding the antigen. The lymphocytes are isolated and fused with a myeloma cell line to form hybridoma cells which are then cultured in conditions which inhibit the growth of the parental myeloma cells but allow growth of the antibody producing cells. The hybridoma may be subject to genetic mutation, which may or may not alter the binding specificity of antibodies produced.

Synthetic antibodies can be made using techniques known in the art (see, for example, Knappik et al, J. Mol. Biol. (2000) 296, 57-86 and Krebs et al, J. Immunol. Meth. (2001) 2154 67-84.

Modifications may be made in the VH, VL or CDRs of the antibody molecules, or indeed in the FRs using any suitable technique known in the art. For example, variable, VH and/or VL domains may be produced by introducing a CDR, e.g. CDR3 into a VH or VL domain lacking such a CDR. Marks et al. (1992) Biol Technology, 10: 779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences of the present invention may be produced.

Alternative techniques of producing antibodies for use in the invention may involve random mutagenesis of gene(s) encoding the VH or VL domain using, for example, error prone PCR (see Gram et al, 1992, P.N.A.S. 89 3576-3580. Additionally or alternatively, CDRs may be targeted for mutagenesis e.g. using the molecular evolution approaches described by Barbas et al 1991 PNAS 3809-3813 and Scier 1996 J Mol Biol 263 551-567.

Having produced such variants, antibodies and fragments may be tested for binding to a USP-17 molecule, for example DUB-3 and for inhibition of the deubiquitinating activity of the molecule.

### Immunoconjugates

In another embodiment of the invention, an antibody molecule for use in the invention may be in the form of an immunoconjugate, comprising an antibody fragment conjugated to an “active therapeutic agent”. The therapeutic agent may be a chemotherapeutic agent or another molecule.

Methods of producing immunoconjugates are well known in the art; for example, see U.S. Pat. No. 5,057,313, Shih et al., Int. J. Cancer 41: 832-839 (1988); Shih et al., Int. J. Cancer 46: 1101-1106 (1990), Wong, Chemistry Of Protein Conjugation And Cross-Linking (CRC Press 1991); Upeslacis et al., “Modification of Antibodies by Chemical Methods,” in Monoclonal Antibodies: Principles And Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering And Clinical Application, Ritter et al. (eds.), pages 60-84 (Cambridge University Press 1995).

Antibody molecules for use in the invention may comprise further modifications. For example the antibodies can be glycosylated, pegylated, or linked to albumin or a nonproteinaceous polymer. The antibody molecule may be in the form of an immunoconjugate.

### Assays

The ability of an agent, for example a small molecule or antibody, to modulate, for example inhibit, the deubiquitinating activity of DUB-3 may be tested using any suitable method. For example inhibitors may be tested for binding to the DUB catalytic domain and for blocking of DUB3 activity. Assays may include the use of ubiquitin conjugated to a target and testing the effect of an agent on cleavage with a recombined DUB3 probe.

The ability of an agent to inhibit cell invasion may be tested using any suitable invasion assay known in the art. For example, invasion assays which measure the movement of cells through an artificial extracellular matrix (ECM) toward a chemoattractant gradient may be employed; the identification of invasion activity would require the ability for the cell to degrade the ECM as well as migrate through it. Such ability may be tested using a modified Boyden chamber as described in the Examples and as, is known in the art. The agent may be tested-using any suitable cell line, for example a MDA-MB-231 cells. An agent may be considered to inhibit cell invasion if it has the ability to inhibit invasion by a statistically significant amount. For example, in one embodiment, an agent for use as the DUB-3 modulator is able to inhibit invasion by at least 10%, for example at least 25%, 50%, 70%, 80% or 90% when compared to an appropriate control antibody.

The ability of an agent to inhibit cell migration may be tested using any suitable migration assay known in the art. For example, migration assays measure the movement of cells toward a chemoattractant gradient may be used. Such ability may be tested using a modified Boyden chamber as described in the Examples and as is known in the art.

The ability of an agent to inhibit cell adhesion may be tested using any suitable adhesion assay known in the art. For example, the ability may be tested by determining the ability of cells to adhere to fibrinogen coated plates as described in the Examples and as is known in the art.

### Treatment

Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and/or vascularisation and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, prostate cervical and ovarian carcinoma, non-small cell lung cancer, hepatocellular carcinoma, lymphomas, including Hodgkin and non-Hodgkin lymphomas, neuroblastoma, melanoma, myeloma, Wilms tumour, and leukemias, including acute lymphoblastic leukaemia and acute myeloblastic leukaemia, astrocytomas, gliomas and retinoblastomas.

As described herein, the present invention is of particular utility in the treatment of Stage 3 and Stage 4 tumours, in which the tumour has metastasised. Many tumour treatments, although successful in the treatment of localised tumours, have little or no therapeutic benefit in the treatment or prevention of metastatic treatment.

As described above the invention provides methods of reducing the number of tumour metastases in an animal with a primary tumour, said method comprising administering a modulator of a USP-17 enzyme, for example DUB-3, to said animal.

The reduction of metastasis may comprise a reduction in the incidence of metastasis i.e. the development of metastases may be inhibited, and/or may comprise the eradication of one or more metastases may be such that the number of pre-existing metastases may be reduced.

The method of the invention may be of particular use in the treatment of metastasis of for example astrocytomas, breast and other tumours where USP17 proteins such as DUB3 are highly expressed.

### Inflammatory Diseases/Infectious Diseases

The invention further finds use in inflammatory diseases, for example in those inflammatory diseases associated with cell invasion. Inflammatory diseases for which the present invention may be use include inflammatory muscle disease, rheumatoid arthritis, allograft rejection, diabetes, multiple sclerosis (MS)/experimental autoimmune encephalomyelitis (EAE), systemic lupus erythematosus (SLE), dermatitis, and asthma.

During spread of infection throughout the body, inflammatory cells invade or extravasate from the circulatory system into normal or diseased tissue. This may lead to chronic inflammation or systemic spread of infection. Therefore the invention may also be used in the treatment of infections such as respiratory infection, infection of the skin, gut or other tissues, or for treatment of conditions such as infectious diseases such as influenza, hepatitis, SARS, etc.

### Pharmaceutical Compositions

USP-17 modulators, for example DUB-3 modulators including antibodies and nucleic acid molecules for use in the present invention may be provided as a pharmaceutical composition. Pharmaceutical compositions a for use in accordance with the present invention may comprise, in addition to active ingredients, a pharmaceutically acceptable excipient, a carrier, buffer stabiliser or other materials well known to those skilled in the art (see, for example, (Remington: the Science and Practice of Pharmacy, 21^{st} edition, Gennaro A R, et al, eds., Lippincott Williams & Wilkins, 2005.). Such materials may include buffers such as acetate, Tris, phosphate, citrate, and other organic acids antioxidants; preservatives; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compounds selected as necessary for the particular indication being treated, preferably with complementary activities that do not adversely affect the activity of the composition of the invention. For example, in the treatment of cancer, in addition to USP-17 modulator the formulation may comprise an additional component, for example a second or further USP-17 modulator, a chemotherapeutic agent, or an antibody, to a target other than a USP-17 protein, for example to a growth factor which affects the growth of a particular cancer.

The active ingredients (e.g. antibody molecules and/or chemotherapeutic agents) may be administered via microspheres, microcapsules liposomes, other microparticulate delivery systems. For example, active ingredients may be entrapped within microcapsules which may be prepared, for example, by coacervation techniques or by interfacial polymerization, for example, hydroxymethylcellulose or gelatine microcapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 21^{st} edition, Gennaro A R, et al, eds., Lippincott Williams & Wilkins, 2005.

Sustained-release preparations may be used for delivery of active agents. Suitable examples of sustained-release preparations include semi-permeable matrices of solid hydrophobic polymers containing the antibody, which matrices are in the form of shaped articles, e.g. films, suppositories or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers, and poly-D-(−)-3-hydroxybutyric acid.

As described above, nucleic acids may also be used in methods of treatment. Nucleic acid for use in the invention may be delivered to cells of interest using any suitable technique known in the art. Nucleic acid (optionally contained in a vector) may be delivered to a patient's cells using in vivo or ex vivo techniques. For in vivo techniques, transfection with viral vectors (such as adenovirus, Herpes simplex I virus, or adeno-associated virus) and lipid-based systems (useful lipids for lipid-mediated transfer of the gene are DOTMA, DOPE and DC-Chol, for example) may be used (see for example, Anderson et al., Science 256:808-813 (1992). See also WO 93/25673).

In ex vivo techniques, the nucleic acid is introduced into isolated cells of the patient with the modified cells being administered to the patient either directly or, for example, encapsulated within porous membranes which are implanted into the patient (see, e.g. U.S. Pat. Nos. 4,892,538 and 5,283,187). Techniques available for introducing nucleic acids into viable cells may include the use of retroviral vectors, liposomes, electroporation, microinjection, cell fusion, DEAE-dextran, the calcium phosphate precipitation method, etc.

The USP-17 modulator, for example an antibody, agent, product or composition, may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

### Dose

USP-17 modulators are suitably administered to an individual in a “therapeutically effective amount”, this being sufficient to show benefit to the individual. The actual dosage regimen will depend on a number of factors including the condition being treated, its severity, the patient being treated, the agents being used, and will be at the discretion of the physician.

### Administration

The USP-17 modulator may be administered simultaneously, separately or sequentially with a chemotherapeutic agent. Where administered separately or sequentially, they may be administered within any suitable time period e.g. within 1, 2, 3, 6, 12, 24, 48 or 72 hours of each other. In one embodiment, they are administered within 6 hours, such as within 2 hours, such as within 1 hour, for example within 20 minutes of each other.

In one embodiment of the invention, the USP-17 modulator and a chemotherapeutic agent are administered in a potentiating ratio, in which the individual components act synergistically.

Synergism may be defined as an RI of greater than unity using the method of Kern (Cancer Res, 48: 117-121, 1988) as modified by Romaneli (Cancer Chemother Pharmacol, 41: 385-390, 1998).

The invention will now be described further in the following non-limiting examples. Reference is made to the accompanying drawings in which:

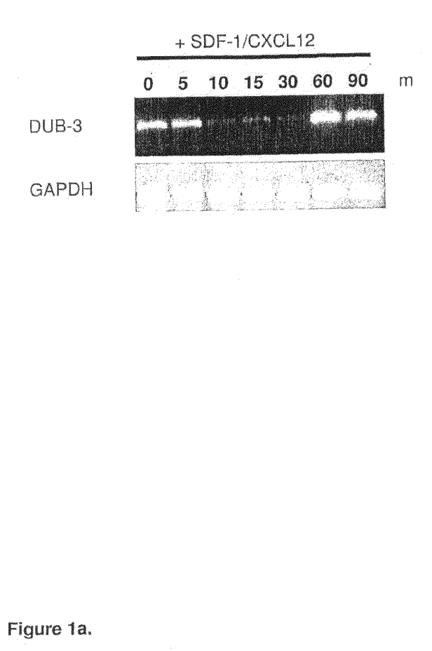

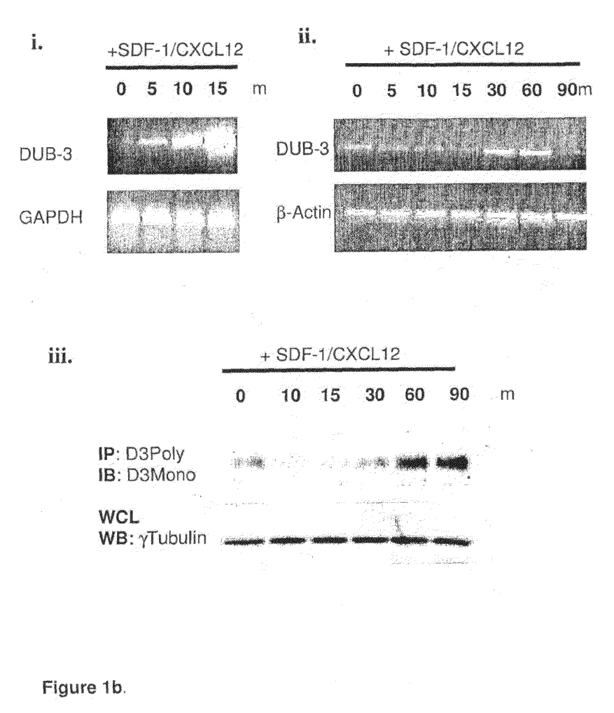

FIG. 1a illustrates the results of RT-PCR of RNA extracted from MDA-MB-231 cells stimulated with 80 ng/ml of SDF-1/CXCL12 wherein the DUB-3 message is down-regulated by the chemokine SDF-1CXCL12.

FIG. 1b illustrates the results of RT-PCR of RNA extracted from Jurkat cells (i) and RNA extracted from PBMCs (ii) and protein extracted from PBMCs (iii) stimulated with 100 ng/ml of SDF-1/CXCL12 wherein the DUB-3 message is up-regulated by the chemokine SDF-1/CDCL12.

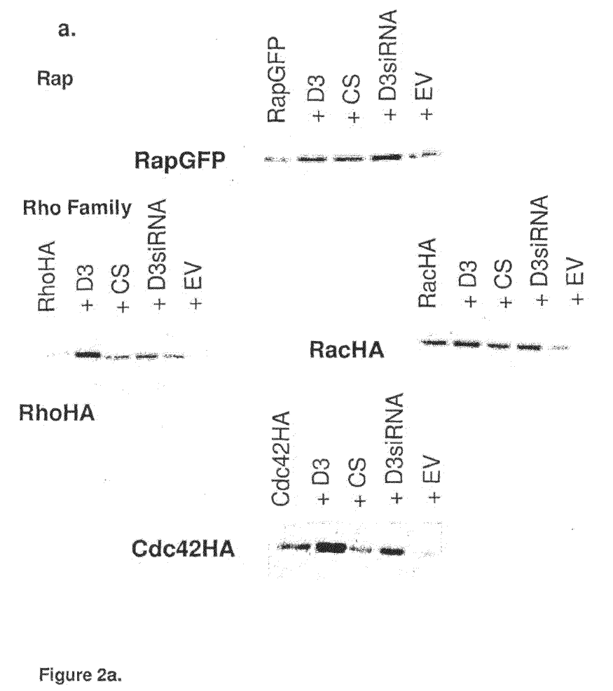

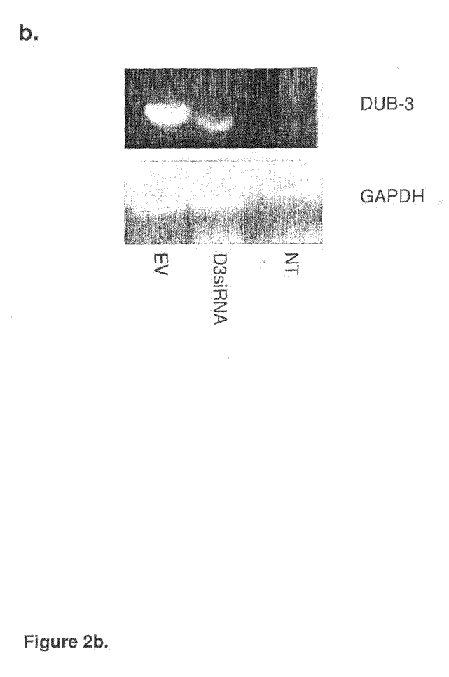

FIGS. 2a-b illustrate that GTPase Protein levels are regulated by Dub-3 in which 2a. shows 293T cells transfected with Rho family GTPases and cotransfected with Dub-3 (D3), Dub-3CS (CS), Dub-3siRNA, and empty vector (EV); and 2b. illustrates knockdown of Dub-3, by Dub-3siRNA in 239T cells.

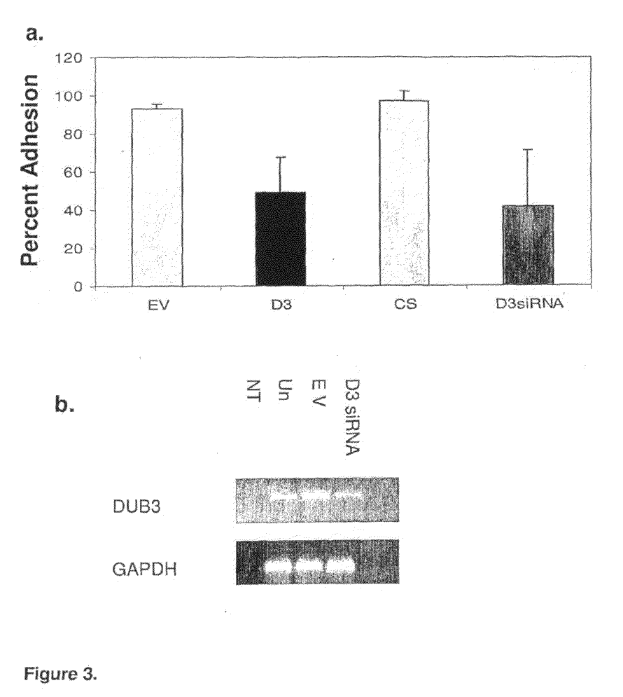

FIG. 3a-b illustrate that deregulation of Dub-3 inhibits cell adhesion in which; 3a. illustrates transfected MDA-MB-231 cells stimulated on fibrinogen coated plates with 10 ng/ml of SDF-1/CXCL12 and the percent cell adhesion determined; and

3b. illustrates Dub-3 RT-PCR of MDA-MB-231 cells transfected with EV and Dub-3siRNA.

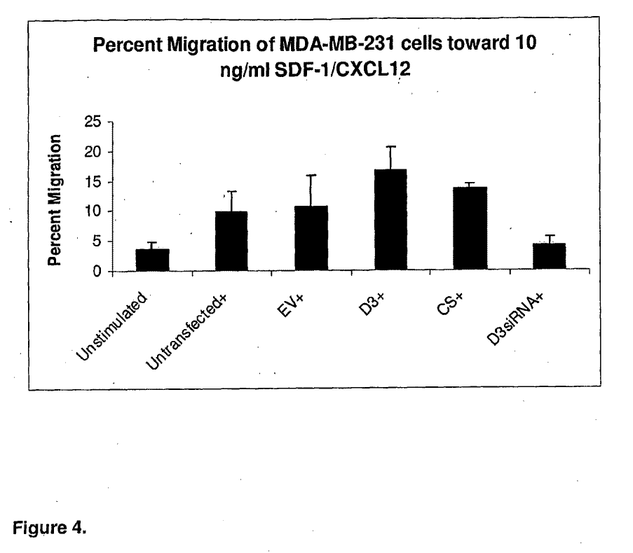

FIG. 4 illustrates percent migration of MDA-MB-231 cells toward 10 ng/ml CXCL12/SDF—Dub-3 knockdown inhibits SDF-1/CXCL12 stimulated chemotaxis—MDA-MB-231 were transfected with EV, DUB3, DUB3CS or DUB3siRNA and subjected to a chemotaxis assay using modified Boyden chambers with cells stimulated for 10 hours with 10 ng/ml SDF/CXCL12.

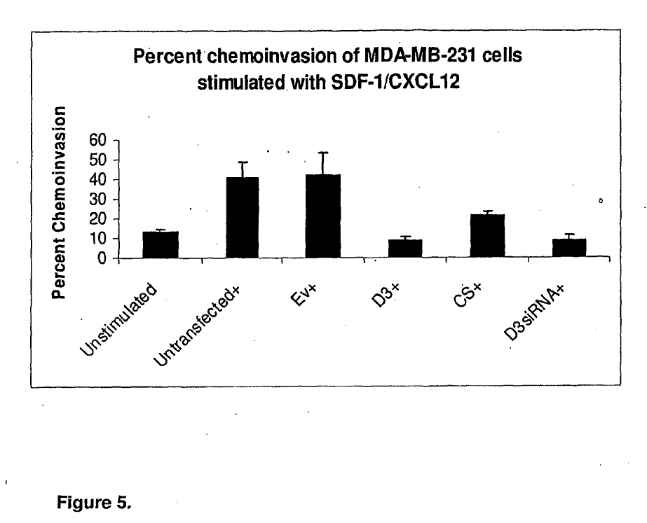

FIG. 5 illustrates percent chemoinvasion of MDA-MB-231 cells stimulated with 10 ng/ml CXCL12/SDF-Deregulation of Dub-3 inhibits SDF-1/CXCL12 driven cell invasion. MDA-MB-231 cells were transfected with EV, Dub-3, Dub-3 C/S, Dub-3 siRNA and stimulated with 10 ng/ml of SDF-1/CXCL12 wherein Dub-3 and Dub-3 siRNA transfected cells had impaired invasion through matrigel.

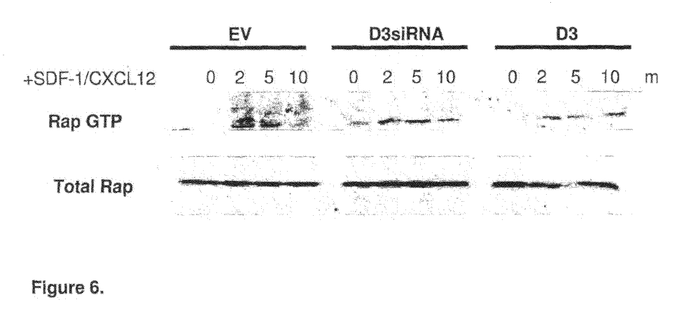

FIG. 6 illustrates that Dub-3 knockdown increases Rap activation in MDA-MB-231 cells. MDA-MB-231 cells transfected with EV, D3 siRNA and D3 were stimulated with 100 ng/ml SDF-1/CXCL12. D3 siRNA cells had increased GTP-bound Rap from a RalGDS—GST fusion protein pulldown.

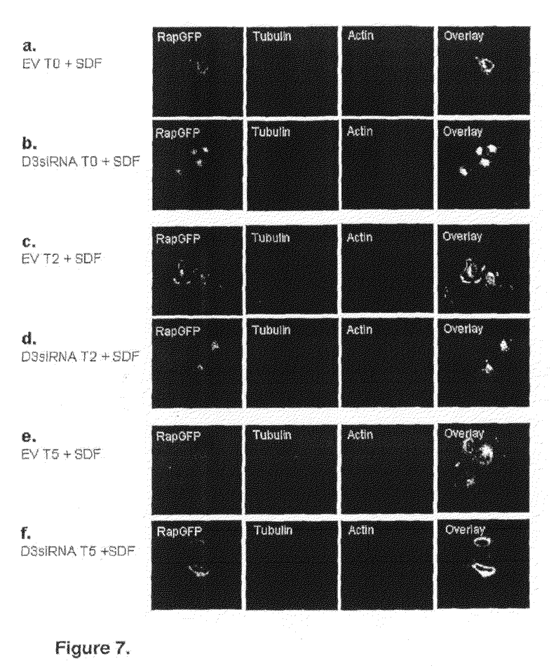

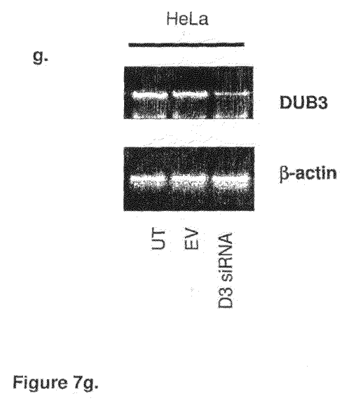

FIGS. 7a-7g illustrate Dub-3 knockdown inhibits SDF-1/CXCL12 stimulated Rap plasma membrane translocation in HeLa cells.

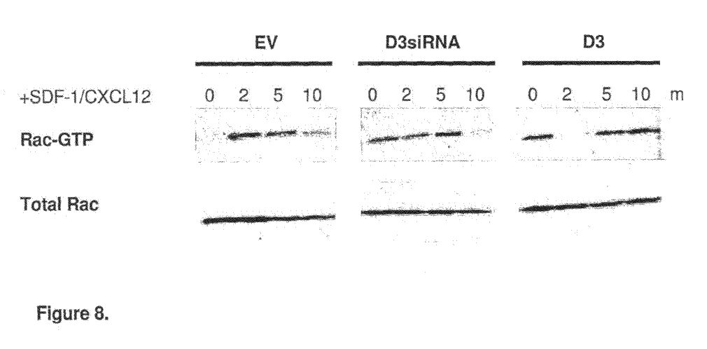

FIG. 8 illustrates that Dub-3 knockdown decreases SDF-1/CXCL12 stimulated Rac activation in MDA-MB-231 cells. MDA-MB-231 cells transfected with EV and D3 siRNA were stimulated with 100 ng/ml SDF-1/CXCL12. D3 siRNA cells had a decrease in GTP-bound Rac from a Pak-GST fusion protein pulldown.

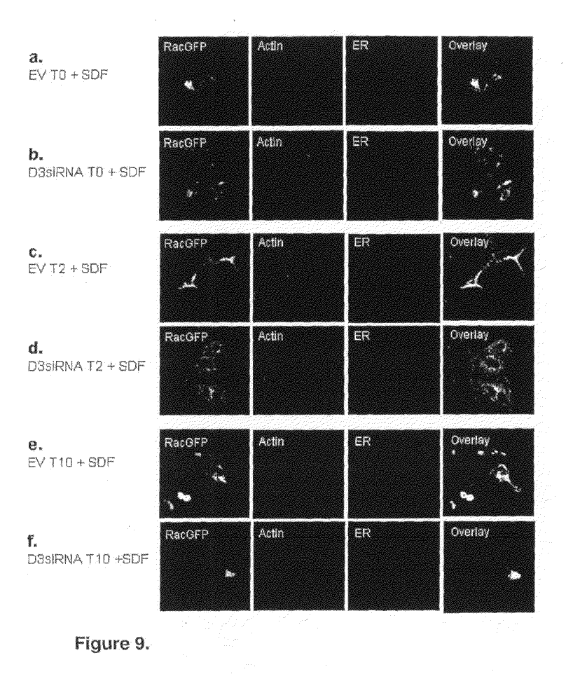

FIGS. 9a-f illustrate Dub-3 knockdown inhibits SDF-1/CXCL12 stimulated Rac plasma membrane translocation in HeLa cells.

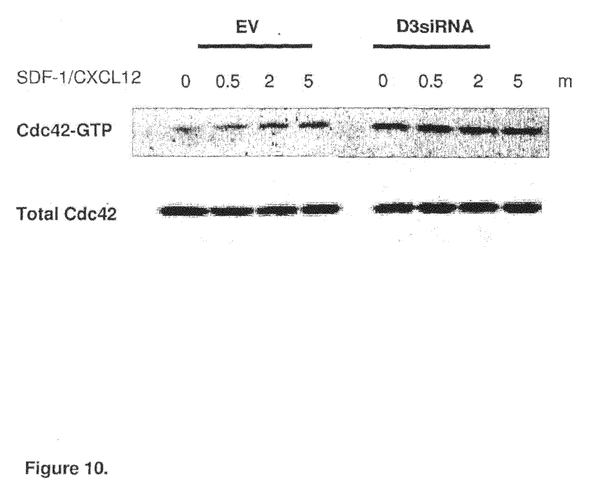

FIG. 10 illustrates the results of a Cdc42GTP pulldown using a PAKGST fusion protein performed from HeLa cells. Knockdown of DUB-3 leads to increased aberrant Cdc42-GTP binding induced by SDF-1/CXCL12. Cells were transfected with EV or DUB3siRNA and stimulated with SDF-1/CXCL12 for 0. 0.5, 2, 5 and 10 m.

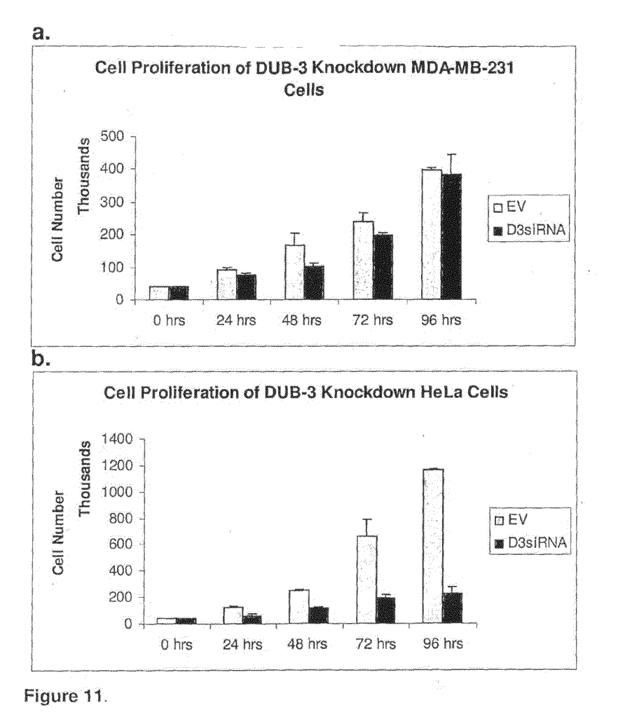

FIG. 11 illustrates Dub-3 knockdown inhibits cell proliferation in MDA-MB-231 cells(a) and HeLa cells (b) in which the cells were transfected with Dub-3siRNA and live cells were counted every 24 hours. The number of Dub-3 knockdown cells was consistently less than empty vector transfected cells.

FIG. 12 illustrates amino acid sequences for DUB-3, DUB-4, DUB-5, DUB-6, DUB-7, DUB-8, DUB-9, DUB-10, DUB-11 or DUB-12.

### EXAMPLES

### Materials and Methods

### Reagents and Antibodies

Rabbit polyclonal DUB-3 antibody was created by Fusion Antibodies (Belfast, UK) as previously described (Burrows J. F., 2004, supra). Other antibodies used included Anti-Rac, and anti-Rap antibodies

### Cell Culture and Transfection

MDA-MB-231 cells, a kind gift from Dr. David Waugh, were grown at 37° C., 5% CO₂ in DMEM (PA) supplemented with 10% fetal calf serum, 1% penicillin/streptomycin, 1% L-glutamine and 1% sodium pyruvate. Jurkat T cells were grown at 37° C., 5% CO₂ in RPMI (PA) supplemented with 10% fetal calf serum, 1% penicillin/streptomycin and 1% L-glutamine. MDA-MB-231 cells were transfected with 3 ug of plasmid DNA at a ratio of 1:3 to Fugene (Roche,) according to manufacturer's instructions.

### Plasmids

The following plasmids were used: Dub-3PDQ, CS-PDQ, D3pSuper, RapGFP, RacGFP, Cdc42GFP, RhoGFP and EVpsuper.

### Chemokine Stimulation and RT-PCR

MDA-MB-231 cells were plated at 1.5×10⁶ cells in 10 cm tissue culture dishes (Nunc) and rested for 12 hours in 0% FCS DMEM. Jurkat cells were rested at 1×10⁶ cells/ml in 2% RPMI for 12 hours. Both pre-plated MDA-MB-231 cells and 9×10⁶ Jurkat cells were stimulated with 100 ng/ml CXCL12/SDF-1 (PeproTech) for indicated times. Cells were then washed in ice cold PBS and RNA was extracted using Stat-60 reagent (Tel-Test Inc, Friendswood, USA). The OneStep RT-PCR kit (Qiagen) was used with the primer sets D1, 5′-CAGTGAATTCGTGGGAATGGAGGACGACTCACTCTAC-3′ and D2,5′-AGTCATCGATCTGGCACACAAGCATAGCCCTC-3′, and GAPDH F 5′-TGATGACATCAAGAAGGTGG-3′ and GAPDH R 5′-TTACTCCTTGGAGGCCATGT-3′ for RT-PCR according to manufacturer's instructions.

### Cell Adhesion Assay.

96 well plates (Nalge Nunc) were coated with 5 μg/ml fibrinogen in PBS and incubated at 4° C. the night before assaying. Cells were removed from the plate and washed several times in Hanks Balanced Salt Solution (HBSS) (PAA) supplement with 10 mM HEPES, 1% bovine serum albumin (BSA) and pH adjusted to 7.5 and counted. Cells were then resuspended in the same HBSS solution and labelled with 10 μm Calcein AM (Molecular Probes, Paisley, UK) by incubating at 37° C. for 30 min. After labelling the cells were washed 3 times and resuspended in 10 mM HEPES, 1% BSA, 1 mM CaCl₂ and 1 mM MgCl₂ HBSS. Cells were added to the coated wells the plate at 50,000 cells per well in 200 μl. The plate was incubated at 37° C. for 1 h then read on a fluorometer Genios Pro (Tecan, Mannedorf/Zurich, Switzerland) (excitation: 485; emission: 530). The wells were then washed 3 times in PBS and the plate was read again. A percentage from the second reading was made from the first reading.

### Cell Migration Assay

MDA-MB-231 cells were transfected as previously described. Cells were trypsinsed and washed in PBS and plated in serum free DMEM at 0.5×10⁶ cells-per top chamber in 500 μl serum free media and 750 μl of serum free media or serum free media containing 10 ng/ml SDF-1/CXCL12 was added to the bottom chamber. Plates were then incubated at 37° C. in 5% CO₂ for 20 hours. After the incubation cells that had not migrated were removed from the top chamber using cotton. Migrated cells on the bottom of the top chamber were fixed for 10 min in methanol and then stained with crystal violet for 20 min. After staining the top chamber was washed in running water and left to air dry for 1 hour. The filters were then destained for 20 min with 300 μl of destaining solution (1 part ethanol: 1 part sodium citrate) and 200 μl of the destain was measured by spectrophotometry at 570 nm (650 nm reference).

### Chemoinvasion Assay

MDA-MB-231 cells were transfected as previously described. Matrigel™ (BD Biosciences, Oxford, UK) was coated and left to dry on 12 mm diameter Transwell (Corning Costar Corp., Cambridge, USA) inserts 12 hours before the chemoinvasion assay began. Cells were then trypsinsed and washed in PBS and plated in serum free DMEM at 0.5×10⁶ cells per top chamber in 500 μl media and 750 μl of serum free media containing 10 ng/ml SDF-1/CXCL12 was added to the bottom. The plates were then incubated at 37° C. with 5% CO₂ for 24 hours. After 24 hours the inserts were removed and the media was discarded. The non-invasive cells were cleaned from the top of the insert using cotton. The inserts were fixed for 10 min in Carnoy's fixative (3 parts methanol: 1 part glacial acetic acid). After the inserts were dried, they were stained in Hoechst 33258 stain (50 ng/ml) for 30 min. After staining the inserts were washed twice in PBS, then the membranes were removed from the insert and mounted on a microscope slide and sealed with a coverslip. The slides were then stored in the dark until viewed. Cells that had invaded were viewed at ×20 using a Nikon Eclipse TE300 fluorescent microscope with a Nikon DXM1200 digital camera. The results were analysed using Luca GF 4.60 by Laboratory Imaging.

### GST-Pull Down Assay

MDA-MB-231 cells were transfected as previously described and rested in 0% FCS DMEM for 12 hours prior to SDF-1/CXCL12 stimulation. Cells were then stimulated with 100 ng/ml of SDF-1/CXCL12 for various times. After stimulation cells were immediately washed with ice cold PBS and lysed in lysis buffer containing 0.5M Tris ph 7.5, 0.1M MgCl2, 0.5M NaCl, 1% Triton and 5% Glycerol with 10 μg/ml leupeptin, 10 μg/ml aprotinin, 1 mM PMSF and 2 mM Na₃VO₄. Lysate was rested on ice for 10 min and spun down at 12,000 RPM for 10 min to pellet the membrane. Lysate was removed and added to either Pak- or RalGDS-GST-fusion protein pre-associated GST beads. The beads, lysate, and fusion protein was left to rotate at 4° C. for 1 hour. The beads were then washed in lysis buffer without inhibitors and then boiled in leamelli buffer with PME then loaded on a 12% polyacrylamide gel.

### Confocal Microscopy

HeLa cells were seeded at 1.5×10⁴ cells per 1.7 cm² well of LabTek II, CC2 treated chamber slides (Nalge Nunc). Cells were transfected as previously described with 0.25 μg of each plasmid using FuGENE6 transfection reagent (Roche). 24 hours after transfection cells were fixed with 4% paraformaldehyde in CBS for 20 minutes at room temperature. The cells were then permeabilized with 0.5% Triton-X 100 in PBS for 5 minutes, washed in PBS and blocked in a blocking solution of 1% BSA, 10% Donkey serum (Jackson ImmunoResearch, Cambridgeshire, UK) in PBS for 1 hour at room temperature. Tubulin was visulaized using an anti-α-tubulin antibody(Molecular Probes) was used at a dilution of 1:200. Anti-calnexin (Abcam, Cambridge, UK) at a 1:100 dilution was used for the detection of the endoplasmic reticulum. Donkey anti-mouse Cy5 or TRITC conjugate (Jackson ImmunoResearch) was used at a 1:200 dilution as a secondary labelled antibody. Each antibody was diluted in blocking soultion and incubated for 1 h at room temperature. Phalloidin Alexa Flour 555 or Rhodamine (Molecular Probes) was used according to manufacturers instructions for the visualization of F-actin. Slides were viewed at 40× using a Leica DBMRE Confocal Microscope (Leica, Milton Keynes, UK) and analyzed using Leica LAS AF software (Leica).

### Proliferation Assay

MDA-MB-231 and HeLa cells were transfected as previously described. 24 h after transfection cells were seeded at 4.2×10⁴ cells per well in 6 well plates (Nalge Nunc). Cells were removed from wells every 24 h, diluted in Trypan Blue and the live cells were counted.

### Results

The inventors have investigated if chemokines specifically could modulate USP-17 e.g. Dub-3 levels. The inventors first stimulated MDA-MB-231 cells with 100 ng/ml of SDF-1/CXCL12 (FIG. 1a), since MDA-MB-231 cells endogenously express CXCR4, the chemokine receptor for SDF-1/CXCL12. Dub-3 message was constitutively expressed at T0 and with stimulation at 10, 15, and 30 min Dub-3 message levels significantly decreased. This result is opposite to cytokine growth factor stimulation which increases Dub-3 message levels upon stimulation (Burrows et al., 2004).

Since MDA-MB-231 cells are an adherent cell line and chemokine stimulation differentially affects adherent and suspension cells, the inventors hypothesized that chemokine stimulation of suspension cells might have a different effect than seen with the MDA-MB-231 cells. Jurkat suspension cells were used since they endogenously express CXCR4 and respond to SDF-1/CXCL12. The Jurkat suspension cells were rested overnight in 2% FCS media, and this abolished Dub-3 message levels that are slightly expressed endogenously (FIG. 1b). The rested cells were then stimulated with 100 ng/ml of SDF-1/CXCL12. Dub-3 message was induced with SDF-1/CXCL12 stimulation as soon as 5 min and peaked at 15 min. Similarly, DUB-3 message was induced in PBMCs with SDF-1/CXCL12 stimulation (FIG. 1b (ii), (iii)). These results suggested that Dub-3 message is regulated by the chemokine SDF-1/CXCL12 and is differentially regulated in various cell types.

The inventors investigated the effect of Dub-3 overexpression and Dub-3 knockdown on Rap and various Rho proteins. FIG. 2a shows 293T cells transfected with the GTPases Rap, RhoA, Rac, and Cdc42 and co-transfected with DUB3, DUB3CS, DUB3siRNA or empty vector (EV). In all GTPases the up- and down-modulation of Dub-3 led to an increase in GTPase protein level compared to untransfected, EV and the DUB3 catalytically inactive co-transfections. These results indicate that the presence or absence of Dub-3 modulation can influence protein levels of several members of the Rho family and Rap. Significant knockdown of Dub-3 in 293T cells transfected with Dub-3siRNA was demonstrated by RT-PCR as shown in FIG. 2b.

Chemokine stimulation is known to induce cell adhesion, cell migration and cell invasion (chemoinvasion). Since chemokine stimulation regulated Dub-3 message expression, the inventors wondered if deregulating Dub-3 could affect chemokine driven functions. They first determined if overexpressing and/or the knockdown of Dub-3 using siRNA could affect MDA-MB-231 cell stimulated adhesion. Previously SDF-1/CXCL12 stimulation of MDA-MB-231 cells have been shown to increase cell adhesion to fibronectin coated plates (Fernandis et al., 2004).

In FIG. 3a MDA-MB-231 cells transfected with DUB3, catalytically inactive DUB3C/S, EV and DUB3siRNA were stimulated with 10 ng/ml of SDF-1/CXCL12 and allowed to adhere to fibrinogen-coated plates. EV and DUB3C/S both showed close to 100% adhesion but the up- and down-modulation of DUB3 by overexpression and knockdown decreased cell adhesion by approximately 50%. DUB3 mRNA was unaffected by EV transfection but was significantly reduced with DUB3 siRNA as shown in FIG. 3b.

Cell adhesion, cell migration and cell invasion driven by chemokines are all known to be mediated by the Rho family of Ras-like small GTPases (Eden et al., 2002; Machesky et al., 1998; Zhuge et al., 2001). The inventors tested whether Dub-3 may affect cell adhesion, migration and invasion by modulating the Rho family of GTPases.

Cell migration is the most associated function of chemokine stimulation. Since deregulation of Dub-3 affected cell adhesion the inventors hypothesized that it would also affect chemotaxis. FIG. 4 shows SDF-1/CXCL12 stimulated migration of MDA-MB-231 cells. Again MDA-MB-231 cells were transfected with EV, Dub-3, Dub-3 C/S, and Dub-3 siRNA. The cells were stimulated with 10 ng/ml of SDF-1/CXCL12 and allowed to migrate through a modified Boyden chamber. Only the siRNA transfected cells showed inhibited chemotaxis approximately equivalent to unstimulated values of less than 5%, whereas the untransfected stimulated cells had a migration rate of approximately 12%.

Chemoinvasion is dependent on cytoskeletal rearrangements, cell adhesion, and matrix degradation for cell migration through the extracellular matrix. Furthermore the formation of the invadopodia, the leading edge of an invading cell, is dependent on firm adhesions to the extracellular matrix. Since Dub-3 deregulation affected both cell migration and cell adhesion Dub-3 deregulated cells were tested for aberrant chemoinvasion. Transfected MDA-MB-231 cells were stimulated with 10 ng/ml of SDF-1/CXCL12 and allowed to invade through BD matrigel coated modified Boyden chambers (FIG. 5). Unstimulated cells had a random invasion rate of approximately 10% whereas stimulated untransfected and EV transfected cells had an invasion rate of approximately 40%. Both Dub-3 overexpressed cells and Dub-3 siRNA cells had impaired chemoinvasion of approximately 10%. These, values were approximately 25% of untransfected and EV tranfected stimulated cells.

Recently, the Ras family member Rap1 has been shown to be a critical GTPase involved in cell adhesion (Katagiri et al 2000; Reedquist et al 2000). Rap1 activated by SDF-1/CXCL12 is known to increase cell adhesion through the subsequent activation of the Rap1 ligand RapL (Katagiri et al., 2003). Moreover, SDF-1/CXCL12 stimulation has previously been shown to activate Rap1 as soon as 30 sec after stimulation (24 Shimonaka, M. 2003). Since FIG. 3 showed up- and down-modulation of DUB3 led to decreased chemokine stimulated cell adhesion the inventors tested whether knockdown or overexpression could affect the activation of Rap1 (FIG. 6). There was a significant increase in Rap1 activation 2 min following SDF-1/CXCL12 treatment in control EV transfected cells compared to negligible Rap1-GTP in EV cells at T0. Dub-3 knockdown cells had increased 4 basal activation of Rap1 as shown in FIG. 6. Moreover, Rap1-GTP levels were sustained 10 min following chemokine stimulation in Dub-3 knockdown cells whereas in EV transfected cells Rap1-GTP was not detectable at 10 min. Conversely, Dub-3 overexpressed cells showed blunted Rap1 activation for all time points. These results therefore suggest Dub-3 regulates the activation of RapGTPase since up- and down-modulation of DUB3 leads to aberrant activation of RapGTPase.

Furthermore, Rap1 has been shown to translocate to the membrane in growth factor stimulated cells (Bivona et al; 2004). Cells become polarized after stimulation by a chemokine gradient and the surface detecting the higher concentration of chemoattractant becomes the leading edge. This is an area of intense actin and tubulin polymerization.

The Rho family of GTPases and the Rap GTPases regulate cytoskeleton polymerization contributing to cell polarity. Cell polarity is also regulated by the RasGTPase Rap1. Where Rap1 knockout cells have inhibited cell polarity and cell adhesion and RapL, ligand for Rap1, deficient cells have inhibited cell polarity and migration (Duchniewicz et al 2006; Katagiri et al 2004). Therefore the inventors investigated the cellular localization of Rap1 in SDF-1/CXCL12 stimulated HeLa cells expressing Rap1-GFP and DUB3siRNA or EV. After incubation in serum free media for 12 hours the cells were stimulated with 50 ng/ml of SDF-1/CXCL12 for periods of 0, 2, 5, and 10 min (FIG. 7). Using confocal microscopy, the cells were analyzed for Rap1 localization as well as F-actin formation and α-tubulin polymerization. F-actin and α-tubulin were chosen as counter-stains to visualize leading edge formation and cell polarization. The EV transfected cells exhibited either a diffuse cytoplasmic localization or a perinuclear localization of Rap1-GFP, and an even distribution of tubulin and F-actin as shown in FIG. 7a. In the Dub-3 knockdown cells, Rap1GFP was observed concentrated perinuclear and there was a strong perinuclear tubulin distribution but neglible peripheral F-actin polymerization (FIG. 7b).

Bivona et al (2004) had previously described Rap1 having a strong peri-nuclear distribution. Two min after SDF-1/CXCL12 stimulation the EV transfected cells showed marked increase in Rap1 plasma membrane localization as well as increased formation of peripheral tubulin and F-actin polymerization (FIG. 7c). Formation of these structures as well as Rap1-GFP membrane localization was not observed in Dub-3 knockdown cells at 2 min, where Rap-1 was still observed with a per-nuclear distribution (FIG. 7d). At 5 min in the EV transfected cells Rap-1 was located at the plasma membrane but less intensely compared to 2 min (FIG. 7e). Rap-1 was still intra-cellularly concentrated around the nucleus in Dub-3 knockdown cells as well the structures associated by cell polarization and leading edge formation were not visible (FIG. 7f). Significant knockdown of DUB3 in HeLa cells transfected with DUB3siRNA was demonstrated by RT-PCR (FIG. 7g).

Such data suggests that Dub-3 influences cell polarity and cytoskeletal rearrangements that are imperative for standard cell adhesion, chemotaxis and chemoinvasion.

The GTPase most commonly associated with cell migration and invasion is the Rho family member Rac. Rac GTPase is known to be activated by chemokines and promote cell polarization and migration through the formation of lamelipodia at the leading edge of a migrating cell. SDF-1/CXCL12 stimulation has previously been shown to activate Rac (Garcia-Bernal et al., 2005). The inventors tested whether Rac-GTP association induced by SDF-1/CXCL12 was affected by siRNA knockdown of Dub-3 or Dub-3 overexpression in MDA-MB-231 cells.

Dub-3 knockdown led to Rac-GTP association at rest: which was not detectable in EV transfected cells (FIG. 8). After SDF-1/CXCL12 stimulation EV cells had a significant increase in Rac-GTP binding. This activation was reduced to background at 10 min in control cells but was sustained at 10 min in the Dub-3 knockdown cells. Cells overexpressing Dub-3 had blunted activation of Rac at all time points examined. Cells overexpressing Dub-3 also had an increase in Rac-GTP association. These results suggest that Dub-3 regulates the activation of RacGTPase since up- and down-modulation of Dub-3 led to aberrant activation of RacGTPase.

Rac has been shown to be an important regulator of actin polymerization leading to the formation of the leading edge and cell polarization in chemotacting cells (Pozo et al., 1999). The cellular localization of Rac1 in SDF-1/CXCL12 stimulated DUB3 knockdown cells was also investigated.

HeLa cells were transfected with Rac-GFP and DUB3siRNA or EV. After resting the cells were-stimulated with 100 ng/ml of SDF-1/CXCL12 for a time course of 0, 2, 5, and 10 min (FIG. 9). Using confocal microscopy the cells were analyzed for Rac localization. F-actin formation and endoplasmic reticulum (ER) localization were co-stained to visualize leading edge formation and cell polarization, respectively. The EV transfected cells had a diffuse cytoplasmic localization of Rac-GFP, an even distribution of F-actin, and a diffuse perinuclear ER (FIG. 9a). In the Dub-3 knockdown cells, RacGFP was concentrated perinuclear and the cells had diminished peripheral F-actin formation (FIG. 9b). Five min after stimulation EV transfected cells showed markedly increased F-actin polmerization and formed cell protrusions and membrane ruffles with concomitantly increased Rac-GFP localization to the cell periphery (FIG. 9c). Furthermore the ER appeared more concentrated and was focused in the direction of movement indicating cell polarization.

Formation of these structures as well as Rac-GFP membrane localization was not observed in DUB3 knockdown cells at 2 min, where Rac was diffusely localized in the cytosol (FIG. 9d). At 10 min in the EV transfected cells there was a dramatic increase in Rac localization to the plasma membrane that co-localized with increased peripheral F-actin as shown in FIG. 9e. At 10 min Rac was diffusely localized in Dub-3 knockdown cells and the structures associated with cell polarization and leading edge formation were not visible (FIG. 9f). Accordingly the results suggest the involvement of Dub-3 in the regulation of Rac activation and cell localization, events important for cytoskeletal rearrangements, cell polarity and subsequent chemotaxis and invasion.

A Cdc42 GTP Pulldown using a PAKGST fusion protein was performed from HeLa cells. Cells were transfected with EV or DUB3siRNA and stimulated with SDF-1/CXCL12 for 0, 0.5, 2, 5, and 10 m. As shown in FIG. 10, knockdown of DUB-3 leads to increased aberrant Cdc42-GTP binding induced by SDF-1/CXCL12.

Since Dub-3 knockdown lead to aberrant activation of the GTPases Rac, Rap and Cdc42 the inventors investigated whether the knockdown of Dub-3 would affect cell proliferation. MDA-MB-231 (FIG. 11a) and HeLa (FIG. 11b) cells were transfected with Dub-3siRNA and EV and a proliferation assay was carried out as described in the material and methods. Dub-3 knockdown led to a significant decrease in MDA-MB-231 cell number compared to EV control. Dub-3 knockdown in HeLa cells dramatically inhibited cell proliferation where EV control proliferated as normal. Such results suggest Dub-3 inhibition leads to inhibition of cell proliferation.

### Discussion

The data shown and discussed above supports USP-17 proteins, for example DUB-3 as a therapeutic target. It has been clearly shown that Dub-3 regulates the dynamic GTPase dependent events that lead to the metastasis of malignant cells. Furthermore it has been demonstrated that by blocking Dub-3 that the critical metastatic events, cell adhesion, cell migration and cell invasion, are inhibited. It has been shown that Dub-3 influences these events by regulating the posttranslational processing, subsequent activation and cellular localization of the essential GTPases involved. Importantly, the inventors have also shown that Dub-3 inhibition significantly reduced malignant cell proliferation, thereby adding to its potential as a therapeutic target.

Here the inventors have shown that expression of the USP 17 protein can be regulated in a chemokine dependent manner and that the deregulation of Dub-3 affects the downstream events of chemokine stimulation: such as cell adhesion, cell migration, chemoinvasion and GTPase activation. The data suggests that Dub-3 activity is tightly regulated for a time specific effect on chemokine driven functions. As well, chemokine stimulated Dub-3 gene regulation, either inhibitory or activatory, may vary according to the morphology of the cell stimulated suggesting that Dub-3 misregulation may have various consequences leading to numerous possible pathological phenotypes.

It is well known that cancerous phenotypes are often the result of misregulated GTPases. As well, invasive cancer cells and cancer metastasis has been shown to be due to misregulated chemokine pathways, leading the inappropriate mobilization of malignant cells (Chan et al., 2005). The Rho GTPases such as Rac, Rho, and Cdc42, known to induce cell adhesion, cell migration and chemoinvasion have been implicated as having major roles in the development of metastatic phenotypes. It is possible that the mis-regulation of these GTPases is the determining factor promoting malignancies and Dub-3 expression and activity may be a determining factor leading to pathogenesis.

Furthermore extracellular matrix (ECM) degradation, a main promoter of cell invasion, is regulated by enzymes that break down ECM components and Matrix Metalloproteinases or MMPS are the enzymes most often associated with ECM degradation leading to an invasive cancer cell phenotype. Recently, the Ras superfamily of small GTPases have been implicated as having a major role in the cell program leading to invasion (Chan et al., 2005). Furthermore, it has been shown that mis-regulation of the Ras superfamily members leads to over activation of MMPs, thereby promoting cell invasion (Bartolome et al., 2004; Deroanne et al., 2005; Zhuge et al., 2001). Therefore, it is possible that USP-17 proteins such as Dub-3 may play a role in the regulation of matrix degradation by modulating Ras superfamily members leading to a pathogenic phenotype.

Here the inventors have shown that the USP-17 proteins such as DUB3 are important for proper activation and regulation of Rho proteins. It is believed that mis-regulated Dub-3 inappropriately activates GTPases subsequently leading to GTPase promotion of invasion and metastasis. The results described herein suggest that Dub-3 has essential roles in chemokine stimulated cell adhesion, migration and chemoinvasion of malignant cells and thus that targeting Dub-3 may have various therapeutic benefits to the treatment of invasive cancers and in controlling inflammatory diseases.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

1. Appels, N. M., Beijnen, J. H. & Schellens, J. H. Development of farnesyl transferase inhibitors: a review. Oncologist 10, 565-578 (2005).
2. Baek, K., Kim, M., Kim, Y., Shin, J. & Choi, H. DUB-1A, a novel deubiquitinating enzyme subfamily member, is polyubiquitinated and cytokine-inducible in B-lymphocytes. J. Biol. Chem. 279, 2368-76 (2004).
3. Baek, K., Mondoux, M., Jaster, R., Fire-Levin, E. & D'Andrea, A. DUB-2A, a new member of the DUB subfamily of hematopoietic deubiquitinating enzymes. Blood 98, 636-42 (2001).
4. Bartolome, R. A. et al. Stromal cell-derived factor-1alpha promotes melanoma cell invasion across basement membranes involving stimulation of membrane-type 1 matrix metalloproteinase and Rho GTPase activities. Cancer Res. 64, 2534-2543 (2004).
5. Bivona, T. G. et al. Rap1 up-regulation and activation on plasma membrane regulates T cell adhesion. J. Cell Biol. 164, 461-470 (2004).
6. Boyartchuk, V. L., Ashby, M. N. & Rine, J. Modulation of Ras and a-factor function by carboxyl-terminal proteolysis. Science 275, 1796-1800 (1997).
7. Burrows, J. et al. DUB-3, a cytokine-inducible deubiquitinating enzyme that blocks proliferation. J. Biol. Chem. 279, 13993-4000 (2004).
8. Casey, P. J. & Seabra, M. C. Protein prenyltransferases. J. Biol. Chem. 271, 5289-5292 (1996).
9. Chan, A. Y. et al. Roles of the Rac1 and Rac3 GTPases in human tumor cell invasion. Oncogene 24, 7821-7829 (2005).
10. Cox, A. D. Farnesyltransferase inhibitors: potential role in the treatment of cancer. Drugs 61, 723-732 (2001).
11. del Pozo, M. A., Vicente-Manzanares, M., Tejedor, R., Serrador, J. M. & Sanchez-Madrid, F. Rho GTPases control migration and polarization of adhesion molecules and cytoskeletal ERM components in T lymphocytes. Eur. J. Immunol. 29, 3609-3620 (1999).
12. Deroanne, C. F. et al. Cdc42 downregulates MMP-1 expression by inhibiting the ERK1/2 pathway. J. Cell. Sci. 118, 1173-1183 (2005).
13. DeSalle, L. et al. The de-ubiquitinating enzyme Unp interacts with the retinoblastoma protein. Oncogene 20, 5538-42 (2001).
14. Duchniewicz, M. et al. Rap1A-deficient T and B cells show impaired integrin-mediated cell adhesion. Mol. Cell. Biol. 26, 643-653 (2006).
15. Eden, S., Rohatgi, R., Podtelejnikov, A. V., Mann, M. & Kirschner, M. W. Mechanism of regulation of WAVE1-induced actin nucleation by Rac1 and Nck. Nature 418, 790-793 (2002).
16. Fernandis, A. Z., Prasad, A., Band, H., Klosel, R. & Ganju, R. K. Regulation of CXCR4-mediated chemotaxis and chemoinvasion of breast cancer cells. Oncogene 23, 157-167 (2004).
17. Garcia-Bernal, D. et al. Vav1 and Rac control chemokine-promoted T lymphocyte adhesion mediated by the integrin alpha4beta1. Mol. Biol. Cell 16, 3223-3235 (2005).
18. Katagiri, K. et al. Rap1 is a potent activation signal for leukocyte function-associated antigen 1 distinct from protein kinase C and phosphatidylinositol-3-OH kinase. Mol. Cell. Biol. 20, 1956-1969 (2000).
19. Katagiri, K., Maeda, A., Shimonaka, M. & Kinashi, T. RAPL, a Rap1-binding molecule that mediates Rap1-induced adhesion through spatial regulation of LFA-1. Nat. Immunol. 4, 741-748 (2003).
20. Katagiri, K. et al. Crucial functions of the Rap1 effector molecule RAPL in lymphocyte and dendritic cell trafficking. Nat. Immunol. 5, 1045-1051 (2004).
21. Li, M. et al. Deubiquitination of p53 by HAUSP is an important pathway for p53 stabilization. Nature 416, 648-53 (2002).
22. Li, Z. et al. Ubiquitination of a novel deubiquitinating enzyme requires direct binding to von Hippel-Lindau tumor suppressor protein. J. Biol. Chem. 277, 4656-62 (2002).
23. Machesky, L. M. & Insall, R. H. Scarl and the related Wiskott-Aldrich syndrome protein, WASP, regulate the actin cytoskeleton through the Arp2/3 complex. Curr. Biol. 8, 1347-1356 (1998).
24. Malliri, A. & Collard, J. G. Role of Rho-family proteins in cell adhesion and cancer. Curr. Opin. Cell Biol. 15, 583-589 (2003).
25. Reedquist, K. A. et al. The small GTPase, Rap1, mediates CD31-induced integrin adhesion. J. Cell Biol. 148, 1151-1158 (2000).
26. Sebti, S. M. & Hamilton, A. D. Farnesyltransferase and geranylgeranyltransferase I inhibitors and cancer therapy: lessons from mechanism and bench-to-bedside translational studies. Oncogene 19, 6584-6593 (2000).
27. Sebti, S. Protein farnesylation: implications for normal physiology, malignant transformation, and: cancer therapy. Cancer Cell 7, 297-300 (2005).
28. Shimonaka, M. et al. Rap1 translates chemokine signals to integrin activation, cell polarization, and motility across vascular endothelium under flow. J. Cell Biol. 161, 417-427 (2003).
29. Wennerberg, K., Rossman, K. L. & Der, C. J. The Ras superfamily at a glance. J. Cell. Sci. 118, 843-846 (2005).
30. Wing, S. Deubiquitinating enzymes—the importance of driving in reverse along the ubiquitin-proteasome pathway. Int. J. Biochem. Cell Biol. 35, 590-605 (2003).
31. Winter-Vann, A. M. & Casey, P. J. Post-prenylation-processing enzymes as new targets in oncogenesis. Nat. Rev. Cancer. 5, 405-412 (2005).
32. Zhu, Y., Carroll, M., Papa, F., Hochstrasser, M. & D'Andrea, A. DUB-1, a deubiquitinating enzyme with growth-suppressing activity. Proc. Natl. Acad. Sci. U.S.A. 93, 3275-9 (1996).
33. Zhu, Y. et al. DUB-2 is a member of a novel family of cytokine-inducible deubiquitinating enzymes. J. Biol. Chem. 272, 51-7 (1997).
34. Zhu, Y., Pless, M., Inhorn, R., Mathey-Prevot, B. & D'Andrea, A. The murine DUB-1 gene is specifically induced by the betac subunit of interleukin-3 receptor. Mol. Cell. Biol. 16, 4808-17 (1996).
35. Zhuge, Y. & Xu, J. Rac1 mediates type I collagen-dependent MMP-2 activation. role in cell invasion across collagen barrier. J. Biol. Chem. 276, 16248-16256 (2001).

1. A method of modulating the activation of a Ras superfamily protein in a biological sample, said method comprising administering to said sample a USP-17 modulator, wherein said Ras superfamily protein is a Rho, Arf, Rab, Ran or Rap family protein.

2. The method according to claim 1, wherein the Ras superfamily protein is a Rho, Arf, Rab, or Ran family protein.

3. The method according to claim 1, wherein the Ras superfamily protein is a Rho family protein.

4. The method according to claim 3, wherein the Rho family protein is RhoA, Rac or Cdc42.

5. The method according to claim 1, wherein the Ras superfamily protein is a Rap protein.

6. The method according to claim 1, wherein the activation of the superfamily protein is chemokine stimulated activation.

7. A method of modulating chemokine induced cell invasion, said method comprising administering a USP-17 modulator.

8. A method of modulating chemokine induced cell adhesion, said method comprising administering a USP-17 modulator.

9. A method of modulating chemokine induced cell migration, said method comprising administering a USP-17 modulator.

10. A method of inhibiting chemokine induced translocation of a Rho, Arf, Rab, Ran and/or a Rap protein in a chemotactic cell, said method comprising administering a USP-17 modulator to said cell.

11. The method according to claim 10, wherein the protein is a Rho protein

12. The method according to any one of claims 5 to 11, wherein the chemokine is CXCL12.

13. A method of reducing the number of tumour metastases in an animal, said method comprising administering a USP-17 modulator to said animal.

14. The method according to claim 13, wherein the animal has at least one primary tumour.

15. The method according to claim 13, wherein the incidence of metastasis is reduced.

16. The method according to claim 13, wherein one or more metastases are eradicated.

17. The method according to claim 13, wherein said animal has a Stage 3 or Stage 4 tumour.

18. The method according to claim 1, wherein said USP-17 modulator inhibits DUB-3 expression and/or DUB-3 activity.

19. A method of modulating the activity or expression of a USP-17 protein in a cell, the method comprising administering a chemokine to said cell.

20. The method according to claim 19, wherein the USP-17 protein is DUB-3.

21. A method of treating an inflammatory disease in an animal in need thereof, said method comprising administering a USP-17 modulator to said animal.

22. The method according to claim 21, wherein the inflammatory disease is an inflammatory disease associated with cell migration.

23. The method according to claim 21, wherein the inflammatory disease is rheumatoid arthritis, allograft rejection, diabetes, multiple sclerosis (MS)/experimental autoimmune encephalomyelitis (EAE), systemic lupus erythematosus (SLE), dermatitis, or asthma.

24. The method according to claim 21, wherein the inflammatory disease is an infectious disease associated with inflammation.

25. The method according to claim 24, wherein the infectious disease is influenza, hepatitis, or severe acute respiratory syndrome (SARS).

26. A method of treating infection associated with cell migration in an animal in need thereof, said method comprising administering a USP-17 modulator to said animal.

27. The method according to claim 26, wherein the infection is respiratory infection, infection of the skin, or infection of the gut.

28. The method according to claim 26, wherein the infection is influenza, hepatitis, or SARS.

29. The method according to claim 21 or 26, wherein said modulator inhibits DUB-3 expression and/or DUB-3 activity.

30. An assay method for identifying a modulator of activation of a Rho, Arf, Rab, or Ran protein said method comprising the steps of:
a) bringing a candidate agent into contact with test cells capable of expressing a USP-17 protein,
b) determining the expression of the USP-17 protein in the presence of the candidate agent in said test cells,
c) comparing the expression of the USP-17 protein in the presence of the candidate agent with control cells not exposed to said candidate agent,
wherein a difference in expression of the USP-17 protein between the control cells and the test cells is indicative that the candidate agent may modulate activation of a Rho, Arf, Rab, or Ran protein.

31. An assay method for identifying a modulator of tumour metastasis, said method comprising the steps of:
a) bringing a candidate modulator into contact with test cells capable of expressing a USP-17 protein
b) determining the expression of the USP-17 protein in the presence of the candidate modulator in said test cells,
c) comparing the expression of the USP-17 protein in the presence of the candidate modulator with control cells not exposed to said candidate modulator,
wherein a difference in expression of the USP-17 protein between the control cells and the test cells is indicative that the candidate modulator may be a modulator of tumour metastasis.

32. An assay method for identifying a modulator of tumour GTPase translocation in a chemotactic cell, said method comprising the steps of:
a) bringing a candidate modulator into contact with test cells capable of expressing a USP-17 protein,
b) determining the expression of the USP-17 protein in the presence of the candidate modulator in said test cells,
c) comparing the expression of the USP-17 protein in the presence of the candidate modulator with control cells not exposed to said candidate modulator,
wherein a difference in expression of the USP-17 protein between the control cells and the test cells is indicative that the candidate modulator may be a modulator of GTPase translocation.

33. The method of claim 32, wherein the GTPase is a Rho protein.

34. The method of claim 32, wherein the GTPase is a Rap1.

35.-40. (canceled)

41. The method according to claim 13 wherein the metastases are metastases of a primary tumour which is promyelocytic leukaemia, chronic myelogenous leukaemia, lymphoblastic leukaemia, Burkitt's lymphoma, cancer of the pancreas, cancer of the lung, cancer of the spleen, cancer of the breast, cancer of the prostate, colorectal cancer, ovarian cancer, brain cancer, bone cancer, or head or neck tumour.

42. The method according to any one of claims 30 to 32, wherein the USP-17 protein is DUB-3.

43. The method according to any one of claims 30 to 32 wherein the difference in expression between the expression of the USP-17 protein between the control cells and the test cells is a reduction in expression in the test cells.

44. The method according to claim 43, wherein the USP-17 protein is DUB-3.

45. (canceled)

46. (canceled)

47. A method of inhibiting adhesion dependent proliferation in cells, said method comprising administering a USP-17 modulator to said cells.

48. The method according to claim 47, wherein the administration of said USP-17 modulator promotes anoikis.

49. The use of a USP-17 modulator for blocking adhesion dependent cell proliferation and/or promoting anoikis.


SEQUENCE LISTING (15 sequences)

SEQ ID NO: 1 - protein, 530 aa, Homo sapiens
MEDDSLYLGGEWQFNHFSKLTSSRPDAAFAEIQRTSLPEKSPLSSEARVDLCDDLAPVARQLAPREKLPLSSRRPAAVGAGLQNMGNTCYENASLQCLTYTPPLANYMLSREHSQTCQRPKCCMLCTMQAHITWALHSPGHVIQPSQALAAGFHRGKQEDAHEFLMFTVDAMKKACLPGHKQVDHHSKDTTLIHQIFGGCWRSQIKCLHCHGIPDTFDPYLDIALDIQAAQSVKQALEQLVKPEELNGENAYHCGLCLQRAPASKTLTLHTSAKVLILVLKRFSDVTGNKLAKNVQYPECLDMQPYMSQQNTGPLVYVLYAVLVHAGWSCHNGHYFSYVKAQEGQWYKMDDAEVTASSITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSTTPTHQESMNTGTLASLRGRARRSKGKNKHSKRALLVCQ

SEQ ID NO: 2 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMEKACLPGHKQVDHHSKDTTLIHQIFGGYWRSQIKCLHCHGISDTFDPYLDIALDIQAAQSVQQALEQLVKPEELNGENAYHCGVCLQRPPASKRLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 3 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQINCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSSTPTHQEAMNTGTLASLRGRTRRSKGKNKHSKRALLVCQ

SEQ ID NO: 4 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 5 - protein, 530 aa, Homo sapiens
MEDDSLYLGGEWQFNHFSKLTSSRPDAAFAEIQRTSLPEKSPLSCETRVDLCDDLAPVARQLAPREKPPLSSRRPAAVGAGLQNMGNTCYVNASLQCLTYKPPLANYMLFREHSQTCHRHKGCMLCTMQAHITRALHIPGHVIQPSQALAAGFHRGKQEDAHEFLMFTVDAMRKACLPGHKQVDRHSKDTTLIHQIFGGYWRSQIKCLHCHGISDTFDPYLDIALDIQAAQSVQQALEQLVKPEELNGENAYHCGVCLQRAPASKTLTLHNSAKVLILVLKRFPDVTGNKIAKNVQYPECLDMQPYMSQQNTGPLVYVLYAVLVHAGWSCHNGHYSSYVKAQEGQWYKMDDAEVTASSITSVLSQQAYVLFYIQKSEWERHSESVSRGREPRALGVEDTDRRATQGELKRDHPCLQAPELDEHLVERATQESTLDHWKFLQEQNKTKPEFNVRRVEGTVPPDVLVIHQSKYKCRMKNHHPEQQSSLLNLSSTTPTDQESMNTGTLASLRGRTRRSKGKNKHSKRALLVCQ

SEQ ID NO: 6 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTTPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQIKCLHCHGISDTFDPYLDIALDIQAAQSVQQALEQLVKPEELNGENAYHSGVCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSALSQQAYVLFYIQKSEWERHSESVSRGREPRALGTEDTDRRATQGELKRDHPCLQAPELDEHLVERATQESTLDHWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 7 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDRWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 8 - protein, 553 aa, Homo sapiens
MEEDSLYLGGEWQFNHFSKLTSSRPDAAFAEIQRTSLPEKSPLSCETRVDLCDDLAPVARQLAPREKLPLSNRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIDKNVQYPECLDMKLYMSQTNSGPLVYVLYAVLVHAGWSCHNGHYFSYVKAQEGQWYKMDDAEVTASSITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSTTPTHQESMNTGTLASLRGRARRSKGKNKHSKRALLVCQWSQWKYRPTRRGAHTHAHTQTHT

SEQ ID NO: 9 - protein, 446 aa, Homo sapiens
MGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQINCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAGWSCYNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 10 - protein, 446 aa, Homo sapiens
MGNTCYVNASLQCLTYTPPLANYMLSREHSQTCHRHKGCMLCTMQAHITRALHNPGHVIQPSQALAAGFHRGKQEDAHEFLMFTVDAMKKACLPRHKQVDHHSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAEWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDHWKFLQEQNKTKPEFNVRKVKGTLPPDVLVIHQSKYKCGMKNHHPEQQSSLLNLSSSTPTHQESMNTGTLASLRGRARRSKGKNKHSKRALLVCQ

SEQ ID NO: 11 - DNA, 37 bp, Artificial
  Chemically synthesised D1 primer
cagtgaattcgtgggaatggaggacgactcactctac

SEQ ID NO: 12 - DNA, 32 bp, Artificial
  Chemically synthesised D2 primer
agtcatcgatctggcacacaagcatagccctc

SEQ ID NO: 13 - DNA, 20 bp, Artificial
  Chemically synthesised GAPDH F primer
tgatgacatcaagaaggtgg

SEQ ID NO: 14 - DNA, 20 bp, Artificial
  Chemically synthesised GAPDH R primer
ttactccttggaggccatgt

SEQ ID NO: 15 - DNA, 38 bp, Artificial
  Hairpin siRNA sequence
gcaggaagatgcccatgaattcatgggcatcttcctgc